(19) United States
(12) Patent Application Publication
(10) Pub. No.: US 2015/0290240 A1
(43) Pub. Date: Oct. 15, 2015

(54) COMPOSITIONS COMPRISING CHALCOGENIDES AND RELATED MATTERS

(71) Applicant: Fred Hulchinson Cancer Research Center, Seattle, WA (US)

(72) Inventors: Mark B. Roth, Seattle, WA (US); Michael L. Morrison, Seattle, WA (US); Akiko Iwata, Seattle, WA (US)

(21) Appl. No.: 14/406,859

(22) PCT Filed: Jun. 12, 2013
(86) PCT No.: PCT/US2013/045395

§ 371 Date: Dec. 10, 2014

Related U.S. Application Data
(60) Provisional application No. 61/798,043, filed on Mar. 15, 2013.
(60) Provisional application No. 61/659,311, filed on Jun. 13, 2012.

(51) Int. Cl. A61K 33/04; A61K 47/20; C01B 19/00

(52) U.S. Cl. CPC A61K 33/04; C01B 19/007; A61K 47/20; A61M 2202/00; A61M 5/14

(57) ABSTRACT

This invention relates to compositions comprising chalcogenides in a reduced form, related methods of producing compositions comprising chalcogenides in a reduced form, devices for delivering a reduced form of a compound to a subject, as well as to methods for treating or preventing injuries or disease using a composition comprising a chalcogenide in a reduced form.

### CROSS REFERENCE TO RELATED APPLICATION

This application claims the benefit of U.S. provisional application No. 61/659,311 filed Jun. 13, 2012, and U.S. provisional application No. 61/798,043, filed Mar. 15, 2013, each of which is incorporated herein by reference in its entirety.

### FIELD OF THE INVENTION

This invention relates to compositions comprising chalcogenides in a reduced form, related methods of producing compositions comprising chalcogenides in a reduced form, devices for delivering a chalcogenide in a reduced form to a subject, as well as to methods for treating or preventing injuriesand diseases using compositions comprising a chalcogenide in a reduced form.

### BACKGROUND OF THE INVENTION

Compounds containing a chalcogen element, i.e., those in Group 6 of the periodic table, but excluding oxides, are commonly termed “chalcogenides” or “chalcogenide compounds.” These elements include sulfur (S), selenium (Se), tellurium (Te) and polonium (Po). Common chalcogenides contain one or more of S, Se, and Te, in addition to other elements.

It has been shown that treatment with a chalcogenide induces stasis of biological matter and protects biological matter from hypoxic and ischemic injury. In these studies, it was demonstrated that hydrogen sulfide (H₂S) gas, a potent inhibitor of oxygen consumption, can reduce metabolism and protect mice and rats from hypoxic injuries (PCT Publication No. WO2005/041655). Although hydrogen sulfide gas has not been typically considered a medical gas, this unexpected result presented exciting possibilities for the treatment or prevention of a number of animal and human diseases, particularly hypoxia and ischemia-related diseases and injuries.

Certain chalcogenide compounds (e.g., H₂S and hydrogen selenide (H₂Se)), are not stable in the presence of oxygen due to their ability to react chemically with oxygen, leading to their oxidation and chemical transformation. Potential oxidizing agents include oxygen, carbon dioxide, and inherent metal impurities that can produce a mixture of oxidation products (e.g., sulfite, sulfate, thiosulfate, polysulfides, dithionate, polythionate, and elemental sulfur or selenium). This chemical transformation of chalcogenide compounds limits their use as pharmaceuticals due to limited stability, limited shelf-life, and the potential for the introduction of oxidation products during manufacture, storage, or use. In addition, commercially available Na₂Se is highly oxidized and contains substantial amounts of elemental selenium, which is not soluble in water, so its injection into animals could block the flow of blood in capillaries.

To provide a pharmaceutical benefit to a cell or subject in need of treatment with a chalcogenide, chalcogenide compositions that are stable, reproducibly manufactured, and designed for standard routes of administration are needed. Clearly, there is a need in the art for stable compositions of chalcogenides, including those containing sulfide or selenide, that may be conveniently administered to patients, in a controlled medical environment, e.g., for treatment of disease, as a treatment in the field during an emergency, or in critical care in response to a catastrophic injury or life-threatening medical event. The present invention meets this need by providing stable compositions of chalcogenides, which are demonstrated herein to protect animals from injury resulting from hypoxic and/or ischemic conditions, as well as other injuries and disease conditions. The present invention also porvides methods of producing stable compositions of chalcogenides.

### BRIEF SUMMARY OF THE INVENTION

The present invention provides methods of producing a stable composition comprising a reduced form of a chalcogenide and related methods for delivering a reduced form of a chalcogenide to a subject, as well as compositions comprising a reduced form of a chalcogenide and related devices for maintaining a compound, e.g., a chalcogenide, in a reduced form. Accordingly, the compositions and methods of the present invention are useful to treat, prevent, inhibit, or reduce injury or disease in a subject, such as ischemia.

Certain embodiments of the invention pertain to a method of producing a stable composition comprising a reduced form of a chalcogenide, wherein the method comprises admixing a chalcogen, or an acid of a chalcogen, and a reducing agent in a reduced oxygen environment under conditions and for a time period sufficient to allow oxidation of a majority of the reducing agent and reduction of a majority of the chalcogen, thereby producing a stable composition comprising a reduced form of a chalcogenide.

In one embodiment, the chalcogen is sulfur or selenium. In another embodiment, the acid of a chalcogen is selenious acid or sodium selenite or elemental selenium.

In certain embodiments, the reducing agent has a reduction potential (Eo) less than or equal to about 0.4 V. In another embodiment, the reducing agent is sodium borohydride (NaBH₄). In one embodiment, the molar ratio of the reducing agent to the chalcogen, or to the acid of a chalcogen, is about 5:1 to about 0.5:1. In a particular embodiment, the molar ratio of the reducing agent to the chalcogen, or to the acid of a chalcogen, is about 2:1. In certain embodiments, the reducing agent is sodium borohydride, and the chalcogen is sulfur or selenium. In certain embodiments, the reducing agent is sodium borohydride, and the acid of a chalcogen is selenious acid. In a related embodiment, the reducing agent is sodium borohydride, the chalcogen is sulfur or selenium, and the acid of a chalcogen is selenious acid. In one embodiment, the reducing agent is sodium borohydride, the chalcogen is selenium, and the molar ratio of sodium borohydride to selenium is about 2:1. In a related embodiment, the selenium is present in an amount of about 1 mM to 1M (79 mg/L to 79 g/L). In yet another embodiment, the sodium borohydride is present in a 1M solution in water.

In one embodiment, the method of producing a stable composition comprising a reduced form of a chalcogenide is performed in an oxygen-free environment. In another embodiment, the method is performed under nitrogen. In a related embodiment, the nitrogen is perfused into the reduced oxygen environment. In a particular embodiment, the nitrogen is perfused at a rate of about 100 cc/min.

In one embodiment of the method of producing a stable composition comprising a reduced form of a chalcogenide, the reduced oxygen environment is a container having an oxygen-free environment. In certain embodiments, the container is a tube or a vial. In another embodiment, the container comprises a closable port of entry. In a related embodiment, the container is a sealable tube comprising a rubber septum, e.g., a Hungate tube. In one embodiment, the perfusion of nitrogen into and out of the tube occurs via two needles that pass through the septum, wherein one of the two needles is used as the port to bring nitrogen into the tube, and wherein the second of the two needles is used to take nitrogen out of the tube.

In another embodiment, the conditions of the method comprise a temperature of about room temperature, or the method is performed at about room temperature. In one embodiment, the method further comprises heating the admixture after the time period. In another embodiment, the time period ends when the admixture solution appears visibly clear. In yet another embodiment, the heating is continued until any observed bubbling ceases. In certain embodiments, the method further comprises cooling the admixture after the heating. In one embodiment, the cooling is performed by placing the admixture on ice. In another embodiment, the cooling is continued until sodium borate precipitates from the admixture solution. In a related embodiment, the method further comprises centrifuging the admixture solution to separate a supernatant from the precipitated sodium borate, and removing the supernatant. In one embodiment, the supernatant comprises the stable composition comprising the reduced chalcogenide.

In certain embodiments, at least 90% of the chalcogenide in the composition is present in a reduced form for at least 1 hour. In one embodiment, the method further comprises acidifying the admixture with an acid, wherein the acid is reducing and not volatile, and bubbling hydrogen selenide gas through a solution, wherein the solution has a pH greater than 3.9. In another embodiment, the acid is phosphorous acid and said solution is phosphate buffered saline (PBS).

In one embodiment, the reduced form of the chalcogenide is in the minus 2 oxidation state. In another embodiment, the reduced form of the chalcogenide is H₂Se, Na₂Se, NaHSe, or HSe-anion.

Certain embodiments relate to a method of producing a stable composition comprising a reduced chalcogenide, wherein the method comprises admixing elemental selenium (Se) or sulfur (S) and sodium hydride in a solution comprising mineral oil or tetrahydrofuran (THF), thereby producing a stable composition comprising sodium hydroselenide or sodium sulfide.

In one embodiment, the method of producing a stable composition comprising a reduced chalcogenide further comprises adding water to the admixture solution, thereby removing sodium hydride. In another embodiment, the solution comprises mineral oil, and the method further comprises removing the aqueous phase of the admixture solution, wherein the reduced chalcogenide is present in the aqueous phase. In one embodiment, the solution comprises THF, and the method further comprises removing the THF by boiling the admixture solution at about 70° C.

Certain embodiments relate to a stable composition comprising a reduced form of a chalcogenide, wherein at least 90% of the chalcogenide in the composition is present in the reduced form for at least one hour when stored at room temperature.

In one embodiment, the stable composition further comprises a pharmaceutically acceptable carrier, diluent or excipient. In another embodiment, the reduced form of a chalcogenide comprises a chalcogen in its −2 oxidation state. In particular embodiments, the reduced form of chalcogenide is optionally H₂Se, Na₂Se, NaHSe, or HSe-anion. In one embodiment, a stable composition comprising a reduced form of a chalcogenide is produced by a method described above. In certain embodiments, the stable composition further comprises one or more of a reducing agent, a tonicity agent, a stabilizer, a surfactant, a lycoprotectant, a polyol, an antioxidant, or a preservative. In a particular embodiment, the elemental chalcogen is selenium or sulfur. In another embodiment, the stable composition further comprises a solvent. In one embodiment, the solvent is water.

Certain embodiments relate to a device for maintaining a compound, e.g., a chalcogenide, in a reduced form. In particular embodiments, the device comprises an oxygen impermeable container.

In one embodiment, the container is glass. In another embodiment, the walls of the container comprise an oxygen impermeable polymer. In a related embodiment, the polymer is selected from the group consisting of: silicon rubber, natural rubber, low density polyethylene (LDPE), polystyrene (PS), polyethylene (PE), polycarbonate (PC), polyvinyl acetate (PVAc), amorphous polyethylene terephthalate (APET), polyvinly chloride (PVC), nylon 6 (Ny6), polyvinyl fluoride (PVF), polyvinylidene chloride (PVdC), polyacetonitrile (PAN), ethylene vinyl alcohol (EVOH), and polyvinyl alcohol (PVA). In another embodiment, the oxygen transmission coefficient of the polymer is less than 10⁻¹⁰ [cm³(STP)/cm/(cm²+s+Pa)]. In one embodiment, the walls of the container comprise multiple layers of one or more oxygen impermeable polymers. In another embodiment, the container comprises a closable port of entry. In yet another embodiment, the container comprises two or more closable ports of entry. In one embodiment, the container is a bottle, a bag, a tube, a vial, or a syringe. In another embodiment, the device is an intravenous bag or a syringe. In one embodiment, the container is a sealable tube comprising a rubber septum, e.g., a Hungate tube.

In another embodiment, the device further comprises a delivery means coupled to the container through a closable port of entry. In one embodiment, the delivery means is configured to intravenously deliver a solution from the container to a subject in need thereof. In one embodiment, the delivery means comprises a needle or cannula. In another embodiment, the delivery means comprises a reduced oxygen environment.

In certain embodiments, the compound is a chalcogenide. In one embodiment, the reduced form of a chalcogenide is H₂Se, Na₂Se, NaHSe, or HSe-anion. In another embodiment, the device comprises any one of the stable compositions described above.

Certain embodiments relate to a method for treating, preventing, inhibiting or reducing an injury or disease or disorder in a subject comprising providing to the subject any one of the stable compositions described herein, thereby treating, preventing or reducing the injury or disease or disorder. In particular embodiments, the injury is a hypoxic injury or an ischemic injury or a reperfusion injury. In certain embodiments, the injury is tissue damage, e.g., due to blood loss, heart attack, or stroke. In particular embodiments, the stable composition comprises a reduced form of a chalcogenide comprising a chalcogen e.g., sulfur or selenium, in a −2 valence state. In particular embodiments, the reduced form of a chalcogenide is H₂Se, Na₂Se, NaHSe, HSe—, H₂S, NaHS, Na₂S, or HS—. In related embodiments, the reduced form of a chalcogenide is produced by a method described herein.

In one embodiment of the method, the stable composition is provided to the subject using any one of the devices described above. In another embodiment, the injury is the result of ischemia or reperfusion. In one embodiment, the injury is an infarct caused by a heart attack or stroke. In another embodiment, the injury is caused by inflammation, heart attack, coronary bypass surgery, ischemia, gut ischemia, liver ischemia, kidney ischemia, stroke, traumatic brain injury, limb ischemia, eye ischemia, sepsis, smoke, burn, or acute lung injury.

Certain embodiments relate to a drug delivery device, comprising a reservoir for containing any one of the stable compositions described above in a reduced form, and a fluid communicator in fluid communication with the reservoir, the fluid communicator configured to maintain at least 90% of the compound, e.g., chalcogenide, in the composition in reduced form during delivery to a patient.

In one embodiment, the reservoir is formed of an oxygen impermeable polymer. In another embodiment, the polymer is selected from the group consisting of silicon rubber, natural rubber, low density polyethylene (LDPE), polystyrene (PS), polyethylene (PE), polycarbonate (PC), polyvinyl acetate (PVAc), APET, polyvinly chloride (PVC), nylon 6 (Ny6), polyvinyl fluoride (PVF), polyvinylidene chloride (PVdC), polyacetonitrile (PAN), ethylene vinyl alcohol (EVOH), and Polyvinyl alcohol (PVA). In one embodiment, the the oxygen transmission coefficient of said polymer is less than 10⁻¹⁰ [cm³(STP)/cm/(cm²+s+Pa)], wherein Pa=pascals; STP=standard temperature and pressure (25 degrees centrigrade and pressure 1 atmosphere); and s=second. In certain embodiments, the reservoir includes multiple layers of oxygen impermeable polymers.

In one embodiment, the reservoir includes a resealable port. In another embodiment, the reservoir includes a plurality of resealable ports. In yet another embodiment, the reservoir is a bottle, a bag, a tube, a vial, or a syringe. In a related embodiment, the device is an intravenous bag or a syringe. In one embodiment, the reservoir is a tubular member, the tubular member having a septum configured to hermetically seal the tubular member. In a particular embodiment, the tubular member is a Hungate tube.

In another embodiment, the fluid communicator is configured to be fluidically coupled to the reservoir through a resealable port. In a related embodiment, the fluid communicator is configured to intravenously deliver the composition from the reservoir to a subject in need thereof. In one embodiment, the fluid communicator includes at least one of a needle and a cannula. In another embodiment, the fluid communicator is disposed in a reduced oxygen or oxygen-free environment. In one embodiment, the composition comprises a reduced form of a chalcogenide. In a particular embodiment, the reduced form of a chalcogenide is H₂Se, Na₂Se or NaHSe.

In certain embodiments, the device is disposed in a reduced oxygen or oxygen-free environment. In one embodiment, the reduced oxygen or oxygen-free environment is within a container, optionally wherein the container is a bag. In another embodiment, the container comprises an oxygen impermeable polymer. In a particular embodiment, the polymer is selected from the group consisting of: silicon rubber, natural rubber, low density polyethylene (LDPE), polystyrene (PS), polyethylene (PE), polycarbonate (PC), polyvinyl acetate (PVAc), amorphous polyethylene terephthalate (APET), polyvinly chloride (PVC), nylon 6 (Ny6), polyvinyl fluoride (PVF), polyvinylidene chloride (PVdC), polyacetonitrile (PAN), ethylene vinyl alcohol (EVOH), and polyvinyl alcohol (PVA). In one embodiment, the oxygen transmission coefficient of the polymer is less than 10⁻¹⁰ [cm³(STP)/cm/(cm²+s+Pa)]. In another embodiment, the walls of the container comprise multiple layers of one or more oxygen impermeable polymers.

In certain embodiments of the invention, the stable cosition comprises glutathione. In certain embodiments, the stable composition comprises glutathione at a concentration of about 1.5 μM to about 10 M, about 15 μM to about 1 M, about 150 μM to about 1 M, about 1.5 mM to about 1 M, about 10 mM to about 500 mM, about 10 mM to about 250 mM, about 100 mM, about 120 mM, about 150 mM, about 170 mM, or about 200 mM.

Certain aspects of the invention relate to a method for reducing, treating, preventing or inhibiting injury, or treating, preventing, reducing or inhibiting a disease or disorder in a mamma, by providing to the mammal the stable composition described above.

In one embodiment, the stable composition is provided to the mammal using the device described above. In another embodiment, the injury is the result of ischemia or reperfusion. In one embodiment, the injury is an infarct caused by a heart attack or stroke. In another embodiment, the injury is caused by inflammation.

Certain aspects of the invention relate to a method of delivering a stable therapeutic composition in a reduced form to a patient in need thereof, wherein the method comprises the steps of containing the therapeutic composition in a reservoir, the reservoir configured to maintain the therapeutic composition in a reduced form; establishing fluid communication between the reservoir in the patient; and delivering a predetermined volume of the therapeutic composition from the reservoir to the patient in an environment substantially free of oxygen.

In certain embodiments of the methods, compositions and devices of the invention, the stable composition further comprises glutathione in addition to the chalcogen. In particular embodiments, it comprises selenide and glutathione. In certain embodiments of the methods, compositions and device of the invention, the reducing agent or antioxidant is glutathione, e.g., glutathione in a reduced state (GSH).

### BRIEF DESCRIPTION OF THE DRAWINGS

FIG. 1 is a bar graph showing that selenium reduced the infarct size in mice following myocardial ischemia and reperfusion. Mice were subjected to LAD ligation for 60 min followed by 2 hours of reperfusion. Sodium selenide (NaHSe) or saline solution was administered I.V. through the femoral vein 5 minutes prior to the reperfusion. The six bars from left to right represent the saline, 0.05 mg/kg NaHSe, 0.2 mg/kg NaHSe, 0.8 mg/kg NaHSe, 1.6 mg/kg NaHSe, and 2.4 mg/kg NaHSe treatment groups. The left panel shows the area at risk (AAR) as a proportion of the left ventricle (LV), and AAR/LV was a similar value in all groups. The middle panel shows infarct size (Inf) as a fraction of the area at risk, and the right panel shows infarct size as a fraction of the left ventricle. NaHSe significantly attenuated infarct size when measured as a fraction of the AAR or LV in dose dependent fashion between 0 and 0.8 mg/kg.

FIG. 2 shows representative mid-myocardial cross section images. FIG. 2A and FIG. 2B show TTC and Evans blue double stained hearts. FIG. 2C and FIG. 2D are images derived from processing the images in FIGS. 2A and 2B in an unbiased way by setting constant thresholds in Photoshop®. FIGS. 2A and 2C depict heart sections from mice receiving saline, and FIGS. 2B and 2D depict heart sections from mice receiving 0.8 mg/kg NaHSe.

FIG. 3 is a bar graph showing that sodium selenide administration reduced plasma cardiac troponin I. Mice were subjected to LAD ligation for 60 minutes followed by 2 hours of reperfusion. At 5 minutes prior to reperfusion, mice were given 0.2 mg/kg NaHSe, 0.05 mg/kg NaHSe, or saline by I.V. injection through the femoral vein. At the end of reperfusion, blood was collected, and plasma cardiac troponin-I (cTnI, ng/mL) was measured by ELISA. Selenide treatment significantly reduced plasma cTnI level in myocardial infarct hearts. *p<0.05 vs. saline by T-test. **p<0.01 vs. saline by Mann Whitney test.

FIG. 4 provides bar graphs showing fractional shortening (FIG. 4A) and LV ejection fraction (FIG. 4B) immediately following 60 minutes of LAD ligation without reperfusion (baseline) and at 48 hours of reperfusion after 60 minutes of LAD ligation. The left bars of each set show the saline treatment group, and the right bars of each set show the NaHSe treatment group.

FIG. 5 provides a micrograph (FIG. 5A) and a bar graph (FIG. 5B) showing that sodium selenide administration decreased the accumulation of neutrophils in the myocardium of animals following heart attack. Neutrophils were counted in sections of hearts of saline (left) and sodium selenide (right) treated animals. Arrows point to neutrophils.

FIG. 6 is a bar graph showing that sodium selenite does not provide benefit in the heart attack model. Mice were given saline, 0.8 mg/kg NaHSe (selenide), or 0.8 mg/kg sodium selenite (selenite) at the point of reperfusion in the heart attack model. Sodium selenide provided benefit, but sodium selenite did not provide benefit to the mice relative to infarct size.

FIG. 7 provides two line graphs showing that selenium is depleted in the blood (FIG. 7B)) and increased in the heart as a function of increasing infarct size. For FIG. 7A: slope=330.0±112.5; Y intercept when X=0.0=1741±2438; X-intercept when Y=0.0=−5.277; and 1/slope=0.003030; for 95% confidence intervals: slope=−153.9 to 813.9; Y intercept when X=0.0=−8747±12230; X intercept when Y=0.0=-infinity to 10.91; and for goodness of fit: R square=0.8115. For FIG. 7B: best-fit values, slope=−96.70±17.36; Y intercept when X=0.0=3278±376.2; X intercept when Y=0.0=33.90; and 1/slope-−0.01034; for 95% confidence intervals: slope=−171 to −22.01; Y intercept when X=0.0=1659 to 4897; X intercept when Y=0.0=28.09 to 76.66; and for goodness of fit: R square=0.9395.

FIG. 8 is a bar graph showing that selenium is mobilized to the heart during reperfusion following ischemia. Relative measure of radioactive selenium in heart tissue was determined in native, 60 minutes of ischemia with no reperfusion (No R), and 60 minutes of ischemia followed by 2 hours of reperfusion (I/R) treated mice. Ischemia and reperfusion hearts had significantly higher levels of selenium than hearts from animals that received ischemia without reperfusion.

FIG. 9 provides photographs demonstrating that glutathione prevents selenide from oxidation. The photographs show samples of 50 mM selenide in either water or 150 mM GSH over the eight minutes immediately following preparation. The oxidized forms of selenide appear dark in the solutions, with the samples in glutathione clearly showing reduced levels of oxidation at each time point.

### DETAILED DESCRIPTION OF THE INVENTION

The present invention is based, in part, on the development of methods for preparing a stable composition comprising a reduced form of a chalcogenide, e.g., selenide or sulfide. The reduced forms of certain chalcogenides, such as selenide and sulfide, are extremely susceptible to oxidation. Accordingly, stable compositions comprising certain reduced forms of chalcogenides have been difficult or impossible to manufacture and/or store, thus limiting their use as pharmaceutical agents in the treatment or prevention of disease or injury.

The present invention is also based, in part, on the surprising and unexpected finding that glutathione stabilizes or prevents oxidation of compounds, such as chalcogenides, including selenide. Thus, the present invention also includes compositions (e.g., stable compositions) comprising glutathione and a compound, such as a chalcogenide, e.g., a reduced form thereof (such as selenide), optionally in combination with an additional active agent, which may be used to treat or prevent diseases or injuries, including, e.g., those associated with hypoxia, ischemia or reperfusion.

The present invention allows the production of stable compositions of reduced forms of chalcogenides, such as selenide and sulfide, which may be used as pharmaceuticals in the treatment of a variety of diseases and injuries, as well as to induce stasis in tissue or an animal. In particular embodiments, the composition is formulated for intravenous administration, administration by infusion, or oral administration.

The compositions of the present invention are particularly advantageous, because they provide stable compositions of reduced active agents, such as chalcogenides. In certain embodiments wherein the compositions comprise glutathione, the glutathione inhibits oxidation of the chalcogenide, thus making the composition more stable are extending its shelf life. In addition, the present invention provides drug delivery devices that maintain the chalcogenide (or another compound or active agent) in its reduced form during administration to a subject, i.e., an animal.

### Definitions and Abbreviations

The words “a” and “an” denote one or more, unless specifically noted.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. In any embodiment discussed in the context of a numerical value used in conjunction with the term “about,” it is specifically contemplated that the term about can be omitted.

Unless the context requires otherwise, throughout the present specification and claims, the word “comprise” and variations thereof, such as, “comprises” and “comprising” are to be construed in an open, inclusive sense, that is as “including, but not limited to”.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

An “increased” or “enhanced” amount is typically a “statistically significant” amount, and may include an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) an amount or level described herein.

A “decreased” or “reduced” or “lesser” amount is typically a “statistically significant” amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

A “composition” can comprise an active agent, e.g., a chalcogen, and a carrier, inert or active, e.g., a pharmaceutically acceptable carrier, diluent or excipient. In particular embodiments, the compositions are sterile, substantially free of endotoxins or non-toxic to recipients at the dosage or concentration employed.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent or emulsifier which has been approved by the United States Food and Drug Administration as being acceptable for use in humans or domestic animals.

The term “biological matter” refers to any living biological material, including cells, tissues, organs, and/or organisms, and any combination thereof. It is contemplated that the methods of the present invention may be practiced on a part of an organism (such as in cells, in tissue, and/or in one or more organs), whether that part remains within the organism or is removed from the organism, or on the whole organism. Moreover, it is contemplated in the context of cells and tissues that homogenous and heterogeneous cell populations may be the subject of embodiments of the invention. The term “in vivo biological matter” refers to biological matter that is in vivo, i.e., still within or attached to an organism. Moreover, the term “biological matter” will be understood as synonymous with the term “biological material.” In certain embodiments, it is contemplated that one or more cells, tissues, or organs is separate from an organism. The term “isolated” can be used to describe such biological matter. It is contemplated that the methods of the present invention may be practiced on in vivo and/or isolated biological matter.

The terms “mammal” and “subject” includes human and non-human mammals, such as, e.g., a human, mouse, rat, rabbit, monkey, cow, hog, sheep, horse, dog, and cat.

“Pharmaceutically acceptable salts” include sulfate, citrate, acetate, oxalate, chloride, bromide, iodide, nitrate, bisulfate, phosphate, acid phosphate, lsomcotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dimtrobenzoate, hydroxybenzoate, methoxybenzoate, methylbenzoate, o-acetoxybenzoate, naphthalene-2-benzoate, isobutyrate, phenylbutyrate, alpha-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, mcotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartarate salts. The term “pharmaceutically acceptable salt” also refers to a salt of compound or active agent, such as a chalcogenide, having an acidic functional group, such as a carboxylic acid functional group, and a base. Suitable bases include, but are not limited to, hydroxides of alkali metals such as sodium, potassium, and lithium, hydroxides of alkaline earth metal such as calcium and magnesium, hydroxides of other metals, such as aluminum and zinc, ammonia, and organic amines, such as unsubstituted or hydroxy-substituted mono-, di-, or tri-alkylamines, dicyclohexylamine, tributylamine, pyridine, N-methyl, N-ethylamine, diethylamine, triethylamine, mono-, bis-, or tris-(2-OH-lower alkylamines), such as mono-, bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine, N-methyl-D-glucamine, and amino acids such as arginine, lysine, and the like. The term “pharmaceutically acceptable salt” also includes a hydrate of a compound of the invention.

The terms “tissue” and “organ” are used according to their ordinary and plain meanings Though tissue is composed of cells, it will be understood that the term “tissue” refers to an aggregate of similar cells forming a definite kind of structural material. Moreover, an organ is a particular type of tissue. In certain embodiments, the tissue or organ is “isolated,” meaning that it is not located within an organism.

The terms “hypoxia” and “hypoxic” refer to an environment with levels of oxygen below normal. Hypoxia occurs when the normal physiologic levels of oxygen are not supplied to a cell, tissue, or organ. “Normoxia” refers to normal physiologic levels of oxygen for the particular cell type, cell state or tissue in question. “Anoxia” is the absence of oxygen. “Hypoxic conditions” are those leading to cellular, organ or organismal hypoxia. These conditions depend on cell type, and on the specific architecture or position of a cell within a tissue or organ, as well as the metabolic status of the cell. For purposes of the present invention, hypoxic conditions include conditions in which oxygen concentration is at or less than normal atmospheric conditions, that is less that 20.8, 20, 19, 18, 17, 16, 15, 14, 13, 12, 11, 10, 9, 8, 7, 6, 5, 4, 3, 2, 1, 0.5, 0%. Alternatively, these numbers could represent the percent of atmosphere at 1 atmosphere of pressure (101.3 kPa). “Anoxia” is the absence of oxygen. An oxygen concentration of zero percent defines anoxic conditions. Thus, hypoxic conditions include anoxic conditions, although in some embodiments, hypoxic conditions of not less than 0.5% are implemented. As used herein, “normoxic conditions” constitute oxygen concentrations of around 20.8% or higher.

The term “buffer” as used herein denotes a pharmaceutically acceptable excipient, which stabilizes the pH of a pharmaceutical preparation. Suitable buffers are well known in the art. Suitable pharmaceutically acceptable buffers include but are not limited to acetate-buffers, histidine-buffers, citrate-buffers, succinate-buffers, tris-buffers and phosphate-buffers. In certain embodiments, the concentration of the buffer is from about 0.01 mM to about 1000 mM, about 0.1 mM to about 1000 mM, about 0.1 mM to about 500 mM, about 0.1 to about 200 mM, about 0.1 to about 100 mM, about 1 mM to about 1000 mM, about 1 mM to about 500 mM, about 1 mM to about 200 mM, about 1 mM to about 100 mM, about 1 mM to about 50 mM, about 2 mM to about 60 mM, about 4 mM to about 60 mM, or about 4 mM to about 40 mM, about 5 mM to about 20 mM, or about 5 mM to about 25 mM.

Pharmaceutically acceptable “cryoprotectants” are known in the art and include without limitation, e.g., sucrose, trehalose, and glycerol. Pharmaceutically acceptable cryoprotectants provide stability protection of compositions, or one or more active ingredients therein, from the effects of freezing and/or lyophilization.

The term “tonicity agent” or “tonicity modifier” as used herein denotes pharmaceutically acceptable agents used to modulate the tonicity of a composition. Suitable tonicity agents include, but are not limited to, sodium chloride, sorbitol, trehalose, potassium chloride, glycerin and any component from the group of amino acids, sugars, as defined herein as well as combinations thereof. In certain embodiments, tonicity agents may be used in an amount of about 1 mM to about 1000 mM, about 1 mM to about 500 mM, about 5 mM to about 500 mM, about 10 mM to about 450 mM, about 20 mM to about 400 mM, about 50 mM to about 300 mM, about 100 mM to about 200 mM, or about 125 mM to about 175 mM. In certain embodiments, a tonicity agent comprises an amino acid present in a composition at about 5 mM to about 500 mM.

The term “stabilizer” indicates a pharmaceutical acceptable excipient, which protects the active pharmaceutical ingredient(s) or agents(s) and/or the composition from chemical and/or physical degradation during manufacturing, storage and application. Stabilizers include, but are not limited to, sugars, amino acids, polyols, surfactants, antioxidants, preservatives, cyclodextrines, e.g. hydroxypropyl-β-cyclodextrine, sulfobutylethyl-β-cyclodextrin, β-cyclodextrin, polyethyleneglycols, e.g. PEG 3000, PEG 3350, PEG 4000, PEG 6000, albumin, e.g. human serum albumin (HSA), bovine serum albumin (BSA), salts, e.g. sodium chloride, magnesium chloride, calcium chloride, and chelators, e.g. EDTA. Stabilizers may be present in the composition in an amount of about 0.1 mM to about 1000 mM, about 1 mM to about 500 mM, about 10 to about 300 mM, or about 100 mM to about 300 mM.

As used herein, the term “surfactant” refers to a pharmaceutically acceptable organic substance having amphipathic structures; namely, it is composed of groups of opposing solubility tendencies, typically an oil-soluble hydrocarbon chain and a water-soluble ionic group. Surfactants can be classified, depending on the charge of the surface-active moiety, into anionic, cationic, and nonionic surfactants. Surfactants may be used as wetting, emulsifying, solubilizing, and dispersing agents for pharmaceutical compositions and preparations of biological materials. In some embodiments of the compositions described herein, the amount of surfactant is described as a percentage expressed in weight/volume percent (w/v %). Suitable pharmaceutically acceptable surfactants include, but are not limited to, the group of polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), or sodium dodecyl sulphate (SDS). Polyoxyethylenesorbitan-fatty acid esters include polysorbate 20, (sold under the trademark Tween 20™) and polysorbate 80 (sold under the trademark Tween 80™). Polyethylene-polypropylene copolymers include those sold under the names Pluronic® F68 or Poloxamer 188™. Polyoxyethylene alkyl ethers include those sold under the trademark Brij™. Alkylphenolpolyoxyethylene ethers include those sold under the tradename Triton-X. Polysorbate 20 (Tween 20™) and polysorbate 80 (Tween 80™) are generally used in a concentration range of about 0.001% w/v to about 1% w/v or about 0.002% w/v to about 0.1% w/v of the total volume of the composition, or alternatively of about 0.003% w/v to about 0.007% w/v. In some embodiments, Tween 80™ is used at about 0.003% w/v, about 0.004% w/v, about 0.0045% w/v, about 0.005% w/v, about 0.0055% w/v, about 0.006% w/v or about 0.007% w/v. In some embodiments, Tween 80™ is used at about 0.005% w/v. In this aspect, “w/v” intends the weight of surfactant per total volume of the composition.

A “lyoprotectant” refers to a pharmaceutically acceptable substance that stabilizes a protein, nucleic acid or other active pharmaceutical ingredient(s) or agent(s) during lyophilization. Examples of lyoprotectants include, without limitation, sucrose, trehalose or mannitol.

A “polyol” refers to an alcohol containing multiple hydroxyl groups, or a sugar alcohol. A sugar alcohol is a hydrogenated form of carbohydrate, whose carbonyl group (aldehyde or ketone, reducing sugar) has been reduced to a primary or secondary hydroxyl group (hence the alcohol). Sugar alcohols have the general formula H(HCHO)ₙ₊₁H, whereas sugars have H(HCHO)ₙHCO.

An “antioxidant” refers to a molecule capable of slowing or preventing the oxidation of other molecules. Antioxidants are often reducing agents, chelating agents and oxygen scavengers such as thiols, ascorbic acid or polyphenols. Non-limiting examples of antioxidants include ascorbic acid (AA, E300), thiosulfate, methionine, tocopherols (E306), propyl gallate (PG, E310), tertiary butylhydroquinone (TBHQ), butylated hydroxyanisole (BHA, E320) and butylated hydroxytoluene (BHT, E321).

A “preservative” is a natural or synthetic chemical that is added to products such as foods, pharmaceutical compositions, paints, biological samples, wood, etc. to prevent decomposition by microbial growth or by undesirable chemical changes. Preservative additives can be used alone or in conjunction with other methods of preservation. Preservatives may be antimicrobial preservatives, which inhibit the growth of bacteria and fungi, or antioxidants such as oxygen absorbers, which inhibit the oxidation of constituents. Examples of antimicrobial preservatives include benzalkonium chloride, benzoic acid, cholorohexidine, glycerin, phenol, potassium sorbate, thimerosal, sulfites (sulfur dioxide, sodium bisulfite, potassium hydrogen sulfite, etc.) and disodium EDTA. Other preservatives include those commonly used in patenteral protein compositions such as benzyl alcohol, phenol, m-cresol, chlorobutanol or methylparaben.

As used in the specification and appended claims, unless specified to the contrary, the following terms have the meaning indicated:

“Mammal” includes humans and both domestic animals such as laboratory animals and household pets, (e.g., cats, dogs, swine, cattle, sheep, goats, horses, and rabbits), and non-domestic animals such as wildlife and the like.

“Optional” or “optionally” means that the subsequently described event of circumstances may or may not occur, and that the description includes instances where said event or circumstance occurs and instances in which it does not.

“Pharmaceutical composition” refers to a formulation of a compound and a medium generally accepted in the art for the delivery of the biologically active compound to mammals, e.g., humans. Such a medium may include any pharmaceutically acceptable carriers, diluents or excipients therefore.

“Sulfide” refers to sulfur in its −2 valence state, e.g., either as H₂S or as a salt thereof (e.g., NaHS, Na₂S, etc.).

“Selenide” refers to selenium in its −2 valence state, e.g., either as H₂Se or as a salt thereof (e.g., NaHSe, Na₂Se, etc.)

“Chalcogenide” or “chalcogenide compounds” refers to compounds containing a chalcogen element, i.e., those in Group 6 of the periodic table, but excluding oxides. These elements are sulfur (S), selenium (Se), tellurium (Te) and polonium (Po). Specific chalcogenides and salts thereof include, but are not limited to: H₂S, Na₂S, NaHS, K₂S, KHS, Rb₂S, CS2S, (NH₄)₂S, (NH₄)HS, BeS, MgS, CaS, SrS, BaS, H₂Se, Na₂Se, NaHSe, K₂Se, KHSe, Rb₂Se, CS₂Se, (NH₄)₂Se, (NH₄)HSe, BeSe, MgSe, CaSe, SrSe, PoSe and BaSe.

As used herein, “oxidation product” refers to products that result from sulfide or selenide chemical transformation, including, e.g., sulfite, sulfate, thiosulfate, polysulfides, dithionate, polythionate, elemental sulfur, selenite, selenate, thioselenate, polyselenides, and elemental selenium. Such products of sulfide or selenide oxidation could occur as a result of processing, manufacturing or storage (e.g., by oxidation).

“Therapeutically effective amount” refers to that amount of a compound of the invention that, when administered to a mammal, preferably a human, is sufficient to effect treatment, as defined below, of a disease or condition in the mammal, preferably a human. The amount of a compound of the invention which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

“Treating” or “treatment” as used herein covers the treatment of the disease or condition of interest, e.g., tissue injury, in a mammal, preferably a human, having the disease or condition of interest, and includes: (i) preventing or inhibiting the disease or condition from occurring in a mammal, in particular, when such mammal is predisposed to the condition but has not yet been diagnosed as having it; (ii) inhibiting the disease or condition, i.e., arresting its development; (iii) relieving the disease or condition, i.e., causing regression of the disease or condition; or (iv) relieving the symptoms resulting from the disease or condition. As used herein, the terms “disease,” “disorder,” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

### A. Stable Reduced Chalcogenide Compositions

In certain embodiments, the present invention includes compositions comprising a reduced form of a chalcogenide and methods useful in their preparation and use. In particular embodiments, a reduced form of a chalcogenide comprises a chalcogen, e.g., sulfur or selenium, in a −2 valence state. In particular embodiments, a composition comprises reduced forms of two or more chalcogenides. In related embodiments, composition is a stable composition. Compositions of the invention may be pharmaceutical compositions.

The compositions include stable compositions of reduced forms of chalcogenides, or chalcogenide compounds or salts or precursors thereof, whose effectiveness as a therapeutic is normally compromised during manufacture and storage as a result of oxidation reactions that produce oxidation products. The compositions of the present invention have increased shelf-life, are easily and reproducibly manufactured, are designed for standard routes of administration, and are advantageous in the treatment and prevention of diseases and conditions where previously or gaseous chalcogenide compositions were considered. The present invention contemplates their use in methods of protecting biological tissue from disease or injury, particularly ischemic or hypoxic injury, as well as methods of treating or preventing injury or disease in a subject.

In certain embodiments of the stable compositions, at least 90% of said chalcogenide in said composition is present in said reduced form for at least one hour either when stored at room temperature, 4° C., 25° C., 40° C. or 50° C. In related embodiments, at least 70%, at least 80%, at least 91%, at least 92%, at least 93%, at least 94%, at least 95%, at least 96%, at least 97%, at least 98%, or at least 99% of the chalcogenide in the composition is present in said reduced form for at least one hour, either when stored at room temperature or when stored at 4° C. In certain embodiments of the stable compositions, at least 90% of said chalcogenide in said composition is present in said reduced form for at least one day, at least one week, at least one month, at least two months, at least four months, at least six months, or at least one year, either when stored at room temperature or when stored at 4° C., 25° C., 40° C. or 50° C. In related embodiments, at least 70%, at least 80%, at least 91%, at least 92%, at least 93%, at least 94%, at least 95%, at least 96%, at least 97%, at least 98%, or at least 99% of the chalcogenide in the composition is present in said reduced form for at least one day, at least one week, at least one month, at least two months, at least four months, at least six months, or at least one year, either when stored at room temperature or when stored at 4° C. In particular embodiments, at least 98% of the chalcogenide in the composition is present in said reduced form for at least one month or at least six months when stored at 4° C. In related embodiments, at least 70%, at least 80%, at least 91%, at least 92%, at least 93%, at least 94%, at least 95%, at least 96%, at least 97%, at least 98%, or at least 99% of the chalcogenide in the composition is present in said reduced form for at least one day, at least one week, at least one month, at least two months, at least four months, at least six months, or at least one year, either when stored at room temperature or when stored at room temperature or 25° C. In particular embodiments, at least 98% of the chalcogenide in the composition is present in said reduced form for at least one month or at least six months when stored at room temperature or 25° C. In related embodiments, at least 70%, at least 80%, at least 91%, at least 92%, at least 93%, at least 94%, at least 95%, at least 96%, at least 97%, at least 98%, or at least 99% of the chalcogenide in the composition is present in said reduced form for at least one day, at least one week, at least one month, at least two months, at least four months, at least six months, or at least one year, either when stored at room temperature or when stored at 40° C. or or 50° C. In particular embodiments, at least 98% of the chalcogenide in the composition is present in said reduced form for at least one month or at least six months when stored at 40° C. or or 50° C.

In particular embodiments, the reduced form of a chalcogenide comprises a chalcogen e.g., sulfur or selenium, in a −2 valence state. In particular embodiments, the reduced form of a chalcogenide is H₂Se, Na₂Se, NaHSe, HSe—, H₂S, NaHS, Na₂S, or HS—. In related embodiments, the reduced form of a chalcogenide is produced by a method described herein.

In particular embodiments, any of the stable compositions described herein comprise a pharmaceutically acceptable carrier, diluent or excipient. Further, any of the stable compositions may comprise one or more of a buffer, a reducing agent, a tonicity agent, a stabilizer, a surfactant, a lycoprotectant, a polyol, an antioxidant, or a preservative.

In particular embodiments, stable compositions may comprise one or more solvents. In particular embodiments, the solvent is water. In particular embodiments, the solvent is a phosphate-buffered saline.

Stable compositions of the present invention may include a chalcogenide or chalcogenide compound or salt or precursor thereof in any desired concentration. The concentration may be readily optimized, e.g., depending upon the type of injury or disease being treated and the route of administration, so as to deliver an effective amount in a convenient manner and over an appropriate time-frame. In some embodiments, the concentration of chalcogenide or chalcogenide compound or salt or precursor thereof is about 0.001 mM to about 5,000 mM, about 1 mM to about 1000 mM, about 10 mM to about 500 mM, about 50 mM to about 500 mM, about 75 mM to about 250 mM, or about 95 mM to 150 mM.

In particular embodiments, the stable composition comprises selenide at a concentration of about 0.1 mM to about 1000 mM, about 1 mM to about 1000 mM, about 5 mM to about 1000 mM, about 10 mM to about 1000 mM, about 10 mM to about 750 mM, about 50 mM to about 500 mM, about 100 mM to about 500 mM, about 10 mM to about 500 mM, 1 mM to about 500 mM, or about 10 mM to about 250 mM.

In particular embodiments the stable composition comprises sulfide at a concentration of about 0.1 mM to about 1000 mM, about 1 mM to about 1000 mM, about 5 mM to about 1000 mM, about 10 mM to about 1000 mM, about 10 mM to about 750 mM, about 50 mM to about 500 mM, about 100 mM to about 500 mM, about 10 mM to about 500 mM, 1 mM to about 500 mM, or about 10 mM to about 250 mM.

In certain embodiments, the concentration of the reduced form of chalcogenide, e.g., selenide or sulfide, in a stable chalcogenide composition of the present invention is about, at least about, or at most about 0.001, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0 mM or M or more or any range derivable therein (at standard temperature and pressure (STP)).

As used herein, the term “%” when used without qualification (as with w/v, v/v, or w/w) means % weight-in-volume for solutions of solids in liquids (w/v), % weight-in-volume for solutions of gases in liquids (w/v), % volume-in-volume for solutions of liquids in liquids (v/v) and weight-in-weight for mixtures of solids and semisolids (w/w) (Remington's Pharmaceutical Sciences (2005); 21st Edition, Troy, David B. Ed. Lippincott, Williams and Wilkins).

In one embodiment, the stable compositions comprise selenide or sulfide measured at 80%-100% (w/v). In one embodiment, the stable compositions comprise selenide or sulfide measured at 90%-100% (w/v). In one embodiment, the stable compositions comprise selenide or sulfide measured at 95%-100% (w/v). In one embodiment, the stable compositions comprise sulfide measured at 98%-100% (w/v).

In particular embodiments, the pH of a stable composition of the present invention is in the range of 3.0-12.0, while in other embodiments, the pH is in the range of 5.0-9.0. The pH of the pharmaceutical composition may be adjusted to a physiologically compatible range. For example, in one embodiment, the pH of the stable composition is in the range of 6.5-8.5. In other embodiments, the stable compositions of the present invention have a pH in the range of 7.5-8.5 or 7.4-9.0.

In particular embodiments, oxygen is present in a stable composition of the present invention at a concentration in the range of 0 μM-5 μM or 0 μM-1 μM or 0 μM-0.1 μM or 0 μM-0.01 μM. In particular embodiments, oxygen is present in the stable composition at a concentration of less than 3 μM, less than 1 μM, less than 0.1 μM, less than 0.01 μM, or less than 0.001 μM.

The pharmaceutical composition of the present invention may further comprise a limited amount of oxidation products. Oxidation products of the present invention include, but are not limited to, selenite, thioselenate, polyselenides, elemental selenium, selenate, sulfite, sulfate, thiosulfate, polysulfides, dithionate, polythionate, and elemental sulfur. In various embodiments, one or more of these oxidation products is present in a stable composition in an amount less than 10%, less than 5.0%, less than 2.0%, less than 1.0%, less than 0.5%, less than 0.2%, less than 0.1%, less than 0.05%, or less than 0.01% (w/v) of the total chalcogenide in the composition.

In one embodiment, a stable composition has an osmolarity in the range of 200-400 mOsmol/L. NaCl may be used as an excipient to adjust osmolality.

In certain embodiments, isotonicity of the stable compositions is desirable as it results in reduced pain upon administration and minimizes potential hemolytic effects associated with hypertonic or hypotonic compositions. Thus, the stable compositions of the invention not only have increased storage stability, but also have the added benefit of substantially reduced pain upon administration when compared with formulations using other more traditional buffer systems consisting of an acid and a salt form of the acid.

In one embodiment, the stable composition is packaged in an impermeable container. “Impermeable container” refers to containers that provide a permanent barrier to the passage of gas molecules. Impermeable containers are known to those skilled in the art and include, but are not limited to, “i.v. bags” or syringes comprising a gas impermeable construction material, or a sealed glass vial. In particular embodiments, the stable composition may be packaged into an impermeable container containing an inert atmosphere, an inert gas, or a noble gas. Noble gas refers to helium (He), neon (Ne), argon (Ar), krypton (Kr), xenon (Xe), and radon (Rn). Inert gas refers to nitrogen (N₂). The term “inert atmosphere” refers to a nitrogen or argon atmosphere in a container. In particular embodiments, the container comprises a reduced oxygen or oxygen-free environment. A “reduced oxygen environment” is an environment having an oxygen concentration of less than 100 parts per million. The stable composition may be packaged in a light-protective vial or container, e.g., amber vials. In one embodiment, the composition is sealed and stored in a glass ampoule.

In some embodiments, stable compositions of the present invention comprise one or more excipients included to prevent or inhibit oxidation of the chalcogenide during storage, where storage is in the range of one to twelve months or longer. In some embodiments, storage is in the range of one to six months. In some embodiments, storage is in the range of three to six months. In some embodiments, storage is in the range of four to five months. Embodiments of the present invention may use a single excipient or a combination of excipients. There are many suitable excipients. Examples include chelators, pH modifying agents, reducing agents, antioxidants, spin-trap agents and preservatives.

In one embodiment, stable compositions of the present invention may optionally contain chelators or chelating agents. Chelating agents yielding soluble metal complexes are also called sequestering agents. A chelating agent typically has at least two functional groups that donate a pair of electrons to the metal, such as —O, —NH₂ or —COO—. Examples of naturally-occurring chelators include carbohydrates, including polysaccharides, organic acids with more than one coordination group, lipids, steroids, amino acids and related compounds, peptides, phosphates, nucleotides, tetrapyrrols, ferrioxamines, ionophores, such as gramicidin, monensin, valinomycin, and phenolics. Examples of synthetic chelators include, but are not limited to, Diethylenetriaminepentaacetic acid (DTPA), Diethylenetriaminepentaacetic acid pentasodium salt (DTPA5), CaDTPAH, dimercaprol (BAL), deferoxamine, desferal, 2,2′-Bipyridyl DimercaptopropanolEthylenediaminotetraacetic acid, Ethylenedioxy-diethylene-dinitrilo-tetraacetic acid (EDTA), CaNa2ethylenediaminetetraacetic acid, Ethylene glycol-bis-(2-aminoethyl)-N,N,N′,N′-tetraacetic acid (EGTA), ionophores, Nitrilotriacetic acid (NTA), ortho-Phenanthroline, Salicylic acid, succimer (meso-2,3-dimercaptosuccinic acid, (DMSA), Triethanolamine (TEA), N-(2-Hydroxyethyl)ethylenediamine-N,N′,N′-triacetic acid Trisodium salt (HEDTA), Nitrilotriacetic acid (NTA). In one embodiment, the synthetic chelator is DTPA. In certain embodiments, the concentration of DTPA is about, at least about, or at most about 0, 0.001, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0 mM or M or any range derivable therein. In one embodiment, the DTPA is in the range of 0.1 mM to 50 mM. In one embodiment, the synthetic chelator consists of DTPA5. In certain embodiments, the concentration of DTPA5 is in the range of (0.0001%-0.1%) (w/v). In another embodiment, DTPA5 is in the range of (0%-1.0%) (w/v). In one embodiment, DTPA5 is in the range of (0% to 0.01%) (w/v). In one embodiment, the synthetic chelator is CaDTPA. In certain embodiments, the concentration of CaDTPA is in the range of (0.0001%-0.1%) (w/v). In one embodiment, CaDTPA is in the range of (0% to 0.01%) (w/v). In another embodiment, CaDTPA is in the range of (0%-1.0%) (w/v). In one embodiment, the synthetic chelator is deferoxamine. In certain embodiments, the concentration of deferoxamine is about, at least about, or at most about 0, 0.001, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0 mM or M, or any range derivable therein. In one embodiment, the deferoxamine is in the range of 0.1 mM to 10 mM. In one embodiment, the synthetic chelator is EDTA. In certain embodiments, the concentration of EDTA is about, at least about, or at most about 0, 0.001, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0 mM or M, or any range derivable therein. In a certain embodiment, EDTA is in the range of 0%-1% (w/v). In another embodiment, EDTA is in the range of 0.0001%-0.1% (w/v). In another embodiment, EDTA is in the range of 0%-1.0% (w/v). In one embodiment, EDTA is in the range of 0% to 0.01% (w/v).

Stable compositions of the present invention may further comprise one or more pH modifying agents. pH modifying agents, include, but are not limited to, inorganic salts, such as zinc carbonate, magnesium carbonate, calcium carbonate, magnesium hydroxide, calcium hydrogen phosphate, calcium acetate, calcium hydroxide, calcium lactate, calcium maleate, calcium oleate, calcium oxalate, calcium phosphate, magnesium acetate, magnesium hydrogen phosphate, magnesium phosphate, magnesium lactate, magnesium maleate, magnesium oleate, magnesium oxalate, sodium chloride, sodium carbonate, sodium bicarbonate, potassium hydroxide, potassium phosphate, sodium bicarbonate, thioglycolic acid, zinc acetate, zinc hydrogen phosphate, zinc phosphate, zinc lactate, zinc maleate, zinc oleate, zinc oxalate, and combinations thereof. Other pH modifying agents include, e.g., acetic acid, fumaric acid, malic acid, nitric acid, phosphoric acid, propionic acid, sulfuric acid, tartaric acid, carbon dioxide, carbonic acid, N-methyl-D-glucamine, 4-(2-hydroxyethyl)-morpholine, Tromethamine, Orotic acid, and hydrochloric acid. In one embodiment, the pH modifying agent is sodium hydroxide.

A pH modifying agent may serve as a buffering agent when it is added to an already acidic or basic solution, which it then modifies and maintains at a new pH (see: The United States Pharmacopeia—National Formulary 29th Edition, (2006) Rockville, Md.; Stahl, P. Wermuth, C. ed. Handbook of Pharmaceutical Salts Properties, Selection and Use. Wiley (2002)).

In certain embodiments, stable compositions of the present invention include one more excipients that are reducing agents, such as, e.g., glutathione (see: U.S. Pat. No. 6,586,404), tris(2-carboxyethyl)phosphine hydrochloride (TSEP), I-cysteine, cysteine or methionine. In one embodiment, the reducing agent is glutathione (see: Vincent et al., Endocrine Reviews (2004) 25:612-628), dithiothreitol (DTT) (Weir et al., Respir and Physiol Biol; (2002) 132:121-30) or dithioerythritol (DTE). In certain embodiments, the concentration of glutathione is about, at least about, or at most about 0, 0.001, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0 mM or M or more or any range derivable therein. In particular embodiments, the stable composition comprises glutathione at a concentration of about 1.5 μM to about 10 M, about 15 μM to about 1 M, about 150 μM to about 1 M, about 1.5 mM to about 1 M, about 10 mM to about 500 mM, about 10 mM to about 250 mM, or about 100 mM, about 120 mM, about 150 mM, about 170 mM, or about 200 mM. In certain embodiments, at least 50%, at least 75%, at least 90%, at least 95%, at least 98%, or at least 99% of the glutathione in a stable composition of the invention is in a reduced state (GSH). In certain embodiments, the concentration of dithiothreitol (DTT), is present at about, at least about, or at most about 0, 0.001, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0 mM or 1 M, or any range derivable therein. In certain embodiments, the reducing agent is dithioerythritol (DTE), which is present at about, at least about, or at most about 0, 0.001, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0 mM or M, or any range derivable therein.

Stable compositions of the present invention may optionally comprise a free radical scavenger or antioxidant. Examples of free radical scavengers or antioxidants include, but are not limited to, ascorbic acid (vitamin C), D-alpha tocopherol acetate, DL-alpha-tocopherol (vitamin E), melatonin, sodium bisulfite, sodium sulfite, sodium metabisulfite, Trolox (6-hydroxy-2,5,7,8-tetramethyl chroman-2-carboxylic acid), Tris(2-Carboxyethyl) phosphine Hydrochloride (TCEP), melatonin, dithionite, pyrosulfite, cysteine, potassium disulfite, sodium thioglycolate, thioethylene glycol, L-threoascobic acid, acetylsalicylic acid, salicylic acid, lecithin, ascorbyl palmitate, butylated hydroxyanidole, ascorbic acid, butylated hydroxyanisole, butylated hydroxyquinone, butylhydroxyanisol, hydroxycomarin, butylated hydroxytoluene, cephalm, ethyl gallate, propyl gallate, octyl gallate, lauryl gallate, propylhydroxybenzoate, trihydroxybutylrophenone, dimethylphenol, lecithin, ethanolamine, meglumine and combinations thereof (see US 2005/0106214). In one embodiment, the anti-oxidant agent is a spin-trap agent. Examples of spin-trap agents include, but are not limited to, N-t-butyl-phenylnitrone (PBN) (see: Kotake, Y., Antioxid Redox Signal (1999) 481), 4-Hydroxy-2,2,6,6-tetramethylpiperidine-1-oxyl (TEMPOL) (Gariboldi, M. B., et al. (2000), Free Radic. Biol. Med. 29:633; Miura, Y., et al. J. Radiat. Res. (Tokyo) (2000) 41:103; Mota-Filipe, H., et al. (1999), Shock 12:255R: 22-41; S: 39-26 2,2,6,6-tetramethylpiperidine-N-oxyl (TEMPO) (see: Lapchak, et al., Stroke (2001) 32:147-53); (disodium-[(tert-butylimino)methyl]benzene-1,3-disulfonate N-oxide (NXY-059) (see: Lapchak et al., CNS Drug Rev (2003) 9:253-62). In some embodiments, the spin-trap agent is TEMPO, which is present in the range of 0 mg/kg-1,000 mg/kg. In some embodiments, the spin-trap agent is TEMPO and is present in the range of 100 mg/kg-1,000 mg/kg. In another embodiment, the spin-trap agent is TEMPO and is present in the range of 0 mg/kg-100 mg/kg.

Stable compositions of the present invention may optionally comprise one or more preservatives. As used herein, the term “preservative” is intended to mean a compound used to prevent the growth of microorganisms. Such compounds by way of example and without limitation, include benzalkonium chloride, benzethonium chloride, benzoic acid, benzyl alcohol, butylated hydroxyanisole (BHA), cetrimonium bromide, cetylpyridinium chloride, chlorobutanol, chlorocresol, cresol, methylparaben sodium, phenol, pheenoxyethanol, phenylethyl alcohol, phenylmercuric acetate, phenylmercuric nitrate, phenylmercuric acetate, thimerosal, metacresol, myristylgamma picolinium chloride, potassium benzoate, potassium sorbate, sodium benzoate, sodium propionate, sorbic acid, thioglycerol, thimerosal, thymol, and methyl, ethyl, propyl or butyl parabens and others known to those of ordinary skill in the art. Such preservatives are used in stable compositions at typical concentrations in accordance with acceptable pharmaceutical practices, such as described. (see: The United States Pharmacopeia—National Formulary 29th Edition, (2006) Rockville, Md.; Remington's Pharmaceutical Sciences (2005) 21st Edition, Troy, D B, Ed. Lippincott, Williams and Wilkins). In a certain embodiment, the preservative is benzyl alcohol and is present in the range of 0%-1.0% (w/v). In one embodiment, the preservative is benzyl alcohol and is present in the range of 0%-0.5% (w/v). In one embodiment, the preservative is phenol in the range of 0%-0.5% (w/v). In a certain embodiment, the preservative is methyl paraben in the range of (0.0%-0.25% (w/v). In a certain embodiment, the preservative is ethyl paraben in the range of 0%-0.25% (w/v). In a certain embodiment, the preservative is propyl paraben in the range of 0%-0.25% (w/v). In a certain embodiment, the preservative is butyl paraben, in the range of 0%-0.4% (w/v). In a certain embodiment, the preservative is benzalkonium chloride in the range of 0%-0.02% (w/v).

The present invention further includes kits comprising stable compositions of the present invention. In certain embodiments, such kits comprise one or more containers to store a stable composition of the present invention. In one embodiment, the stable composition is stored in the container under an inert or noble gas, and the container is a sealed and has an oxygen impermeable light-protective container (e.g., an amber vial). In certain embodiments, a container is an oxygen-impermeable container.

### B. Methods of Preparing Stable Reduced Chalcogenide Compositions

In certain embodiments, the present invention provides methods of preparing a stable composition comprising a reduced form of a chalcogenide, e.g., a chalcogenide comprising selenium or sulfur is the −2 valence state, e.g., selenide or sulfide.

Certain chalcogenide compounds (e.g., hydrogen sulfide, hydrogen selenide), are not stable in the presence of oxygen due to their ability to react chemically with oxygen, leading to their oxidation and chemical transformation. Accordingly, oxygen may be removed from liquids or solutions used in these methods, using methods known in the art, including, but not limited to, application of negative pressure (vacuum degasing) to the liquid or solution, or contacting the solution or with a reagent which causes oxygen to be bound or “chelated”, effectively removing it from solution. In general, methods of preparing stable compositions of the present invention include limiting oxygen content in each aspect of manufacturing and storage.

In one embodiment, the present invention includes a method of producing a stable composition comprising a reduced form of a chalcogenide, said method comprising: admixing a chalcogen, or an acid of a chalcogen, and a reducing agent in a reduced oxygen environment under conditions and for a time period sufficient to allow oxidation of a majority of the reducing agent and reduction of a majority of the chalcogen. In certain embodiments, the conditions include a temperature of about room temperature. In particular embodiments, the time period is about one hour, about two hours, about three hours, or overnight, e.g., about 12 hours. In particular embodiments, the time period is finished or ends when the admixture solution appears visibly clear or bubbling (due to hydrogen generation from the borohydride reacting with water) ceases or is no longer observed.

In particular embodiments, the chalcogen is sulfur or selenium, and the reduced form of a chalcogenide comprises sulfur or selenium in the −2 oxidation state. In certain embodiment, the acid of a chalcogen is selenous acid or sodium selenite or elemental selenium, and the reduced form of a chalcogenide comprises sulfur or selenium in the −2 oxidation state.

In particular embodiments, the reducing agent has a reduction potential (E°) less than or equal to about 0.4 V. In one embodiment, the reducing agent is sodium borohydride (NaBH₄). In certain embodiments, the molar ratio of the reducing agent to the chalcogen, or the acid of a chalcogen, is about 5:1 to about 0.5:1, or about 3:1 to about 1:1. In one embodiment, the molar ratio of the reducing agent to the chalcogen, or the acid of a chalcogen, is about 2:1.

In particular embodiments, the reducing agent is sodium borohydride, said chalcogen is sulfur or selenium, and said acid of a chalcogen is selenious acid. In one particular embodiment, the reducing agent is sodium borohydride, the chalcogen is selenium, and the molar ratio of sodium borohydride to selenium is about 2:1. In particular embodiments, the selenium is present in an amount of about 1 mM to about 10 M, or about 1 mM to 1M (79 mg/L to 79 g/L). In particular embodiments, the sodium borohydride is present in a 1M solution in water.

In certain embodiment, the method is performed in a reduced oxygen or oxygen-free environment. In certain embodiments, the method is performed under an inert or noble gas. In certain embodiments, the method is performed under nitrogen. For example, the nitrogen may be perfused into said reduced oxygen environment. In certain embodiments, the nitrogen is perfused at a rate of about 100 cc/min. In particular embodiments, the reduced oxygen environment is a container having an oxygen-free environment. In particular embodiments, the container is a syringe, i.v. bag, tube or a vial. In certain embodiments, the container comprises a closable port of entry or a resealable port. In One embodiment, the container is a sealable tube comprising a rubber septum, e.g., a Hungate tube.

In particular embodiments of this method, the perfusion of nitrogen into and out of the container, e.g., tube, occurs via two needles that pass through the septum of the container, wherein one of the two needles is used as the port to bring nitrogen into the tube, and wherein the second of the two needles is used to take nitrogen out of the tube.

In particular embodiments, the method further includes heating the admixture after said time period. In particular embodiments, the heating is continued until any observed bubbling ceases. The methods may also include cooling the admixture after the heating. Cooling may be performed, e.g., by placing the admixture on ice. In certain embodiments, cooling is continued until sodium borate precipitates from the admixture solution.

These methods may also further comprise centrifuging the admixture solution to separate a supernatant from the precipitated sodium borate, and removing said supernatant, wherein the supernatant comprises said stable composition comprising said reduced chalcogenide.

In particular embodiments, the methods comprise acidifying the admixture with an acid, wherein said acid is reducing and not volatile, and bubbling hydrogen selenide gas or hydrogen sulfide gas through a solution, wherein the solution has a pH greater than 3.9. Without wishing to be bound to any particular theory, it is believed that if a non volatile acid (e.g. phosphorus acid) is added to a solution of reduced chalcogenide that may have unwanted non volatile compounds in the solution, it will lower the pH to a value less than the pK for the chalcogenide, thus producing a gas form (e.g. H₂S or H₂Se). The gas form is able to be blown out of the solution by passing nitrogen through the mixture and carried to a second solution that has a pH above the pK rendering the chalcogenide into an ionic form that is no longer a gas (i.e. trapping the chacogenide).

In certain embodiment, the acid is phosphorous acid and said solution is phosphate buffered saline (PBS). This permits the hydrogen selenide or hydrogen sulfide gas to be trapped in a vessel.

In particular embodiments of any of the methods of producing a stable composition described herein, the reduced form of the chalcogenide is in the minus 2 oxidation or valence state. In certain embodiments, the reduced form of the chalcogenide is H₂Se, Na₂Se, NaHSe, or HSe-anion.

In another embodiment, the present invention includes another method of producing a stable composition comprising a reduced form of a chalcogenide, the method comprising admixing elemental selenium (Se) or sulfur (S) and sodium hydride in a solution comprising mineral oil or tetrahydrofuran (THF), thereby producing a stable composition comprising sodium hydroselenide or sodium sulfide. In certain embodiments, the method also comprises adding water to the admixture solution, thereby removing sodium hydride. In particular embodiments wherein the solution comprises mineral oil, the method further comprises removing the aqueous phase of the admixture solution, wherein the reduced chalcogenide is present in the aqueous phase. In particular embodiments wherein the solution comprises THF, the method further comprises removing said THF by boiling said admixture solution at about 70° C.

In various embodiments, methods of preparing stable compositions of the present invention further include adjusting the pH of the composition. In certain embodiments, the pH is adjusted by the addition of one or more of hydrogen chloride, carbon dioxide, nitrogen, or hydrogen sulfide. In another embodiment, the pH is adjusted by dissolving nitrogen, carbon dioxide, hydrogen selenide, or hydrogen sulfide into the composition or any combination thereof.

In certain embodiments, the methods of producing a stable composition of the invention comprise admixing glutathione with the chalcogen and reducing agent, or adding it to the stable composition. Glutathione exists in reduced (GSH) and oxidized (GSSG) states.

Once produced, in various embodiments, the stable composition is stored in an impermeable container, e.g., an oxygen impermeable container. This is particularly desirable to prevent oxidation of the reduced form of chalcogenide. Impermeable containers are known to those skilled in the art and include, but are not limited to, “i.v. bags” comprising a gas impermeable construction material, or a sealed glass vial. In particular embodiments, the impermeable container comprises an oxygen impermeable material having an oxygen transmission coefficient less than 10⁻¹⁰ [cm³(STP)/cm/(cm²+s+Pa)], wherein STP=standard temperature and pressure (25 degrees centigrade and pressure 1 atmosphere); PA=pascals, and s=second. For example, the walls of the container may comprise a layer of an oxygen impermeable polymer. Exemplary oxygen impermeable polymers include but are not limited to: silicon rubber, natural rubber, low density poly ethylene (LDPE), polystyrene (PS), polyethylene (PE), polycarbonate (PC), polyvinyl acetate (PVAc), amorphous polyethylene terephthalate (APET), polyvinly chloride (PVC), nylon 6 (Ny6), polyvinyl fluoride (PVF), polyvinylidene chloride (PVdC), polyacetonitrile (PAN), ethylene vinyl alcohol (EVOH), and polyvinyl alcohol (PVA). In certain embodiments, the oxygen transmission coefficient of said polymer is less than 10⁻¹⁰ [cm³(STP)/cm/(cm²+s+Pa)]. In particular embodiments, the walls of the container comprise multiple layers of one or more oxygen impermeable polymers.

In additional embodiments, the container comprises one or more resealable or closable ports of entry. In certain embodiments, the container comprises two or more resealable or closable ports of entry. As noted, in specific embodiments, the container is a bottle, a bag, a tube, a vial, or a syringe. In certain embodiments, the container is an intravenous bag or a syringe. In particular embodiments, the container is a sealable tube comprising a rubber septum, e.g., a Hungate tube.

In related embodiments, the device further comprises a delivery means coupled to the container through a resealable or closable port of entry. In particular embodiments, the delivery means is configured to intravenously deliver a solution from the container to a subject in need thereof. For example, the delivery means may be a needle or a cannula. In certain embodiments, the delivery means comprises a reduced oxygen or oxygen-free environment or is present under a reduced oxygen or oxygen-free environment.

In particular embodiments of storage or delivery devices of the present invention, the compound is a chalcogenide. In certain embodiments, the compound is a reduced form of a chalcogenide, e.g., H₂Se, Na₂Se, NaHSe, or HSe-anion or others described herein. In certain embodiments, the device comprises a stable composition of the present invention.

To prevent exposure to air in the gas-tight storage container, an inert or noble gas, such as nitrogen or argon, may be introduced into a container containing a stable composition of the present invention prior to closure.

In other related embodiments, stable compositions are stored in a light-resistant or a light-protective container or vial, such as an amber vial. The composition may be packaged in a glass vial. It may be filled to a slight over-pressure in an inert atmosphere, e.g., nitrogen, to prevent/slow oxidative breakdown of the composition, and may be contained in a form such that ingress of light is prevented, thereby preventing photochemical degradation of the composition. This may be achieved using an amber vial. Additional container systems that permit a solution to be stored in an oxygen-free environment are known, as many intravenous solutions are sensitive to oxygen. For example, a glass container that is purged of oxygen during the filling and sealing process may be used. In another embodiment, flexible plastic containers are available that may be enclosed in an overwrap to seal against oxygen. Basically, any container that prevents oxygen from interacting with the stable composition may be used (see, e.g., U.S. Pat. No. 6,458,758). In one embodiment, the container includes one or more oxygen scavenger. For example, the oxygen scavenging composition can be applied as a coating or lining upon the inside surface of the product supporting or retaining means to function as a barrier to oxygen permeation (see, e.g., U.S. Pat. No. 5,492,742).

### C. Methods of Using Stable Reduced Chalcogenide Compositions

In certain embodiments, a stable compositions of the present invention is used to treat or preven or inhibit an injury or a disease in a biological material, e.g., a subject, e.g., a mammal, such as a human. In particular embodiments, a stable composition of the present invention is used to treat a subject (or biological material) prior to, during, or after exposure to an ischemic or hypoxic condition, or reperfusion. Biological material may be treated in vivo or ex vivo. In specific embodiments, a stable composition of the invention is used to treat or prevent injury caused by a heart attack or infarct resulting from a heart attack.

In one embodiment, stable compositions of the present invention are used to treat subjects who have undergone, are undergoing, or who are susceptible to a disease, injury, trauma or critical care treatment. In particular embodiments, the injury may be caused by external insults, such as burns, wounds, amputations, gunshot wounds, or surgical trauma, abdominal surgery, prostate surgery, internal insults, such as septic shock, stroke or cardiac arrest, heart attack, e.g., that result in the acute reduction in circulation, or reductions in circulation due to non-invasive stress, such as exposure to cold or radiation. On a cellular level, injury often results in exposure of cells, tissues and/or organs to hypoxia, thereby resulting in induction of programmed cell death, or “apoptosis.” In particular embodiments, injury results from reperfusion of oxygen into cells, a tissue, an organ, or a mammal following an ischemic event.

In one embodiment, the present invention contemplates contacting tissues, organs, limbs and even whole organisms with an effective amount of a stable composition of the present invention as a way of protecting them from a detrimental effect of injury, or reducing a detrimental effect of an injury. The present invention also contemplates methods for inducing tissue regeneration and wound healing by inhibition/prevention/delay of biological processes that may result in delayed wound healing and tissue regeneration. In this context, in scenarios in which there is a substantial wound to the limb or organism, contacting the biological matter with a stable composition of the present invention aids in the wound healing and tissue regeneration process by managing the biological processes that inhibit healing and regeneration. In addition to wound healing, methods of the invention can be implemented to prevent, inhibit or treat trauma such as cardiac arrest or stroke, and hemorrhagic shock. The invention has importance with respect to the risk of trauma from emergency surgical procedures, such as thoroacotomy, laparotomy, and splenic transaction or cardiac surgery, aneurysm, surgery, brain surgery and the like.

In certain embodiments, methods of the present invention can be implemented to enhance survivability or treat, inhibit or prevent ischemic injury or treat, inhibit or prevent reperfusion injury, e.g., resulting from cardiac arrest or stroke. Accordingly, in one embodiment, the present invention includes methods of enhancing survivability or reducing ischemic or reperfusion injury in a subject suffering from or at risk of cardiac arrest or stroke, comprising providing an effective amount of a stable composition of the invention to the patient before, after, or both before and after myocardial infarction, cardiac arrest or stroke. “Ischemia” refers to a restriction in blood supply to tissues, causing a shortage of oxygen and glucose needed for cellular metabolism. It can be caused by problems with blood vessels, leading to damage to the affected tissue. In certain embodiments, it results from vasoconstriction, thrombosis, myocardial infarct, stroke, or embolism. As used herein, “reperfusion injury” refers to tissue damage resulting from blood returning to tissue after a period of ischemia or lack of oxygen. It is believed that the absence of oxygen and nutrients from blood during the ischemic period creates a condition in which the restoration of circulation results in inflammation and oxidative damage through the induction of oxidative stress. In certain embodiments, the method results in a reduced infarct size as compared to the size in the absence of treatment with the stable composition.

In certain embodiments, methods of the present invention include pre-treating a biological material, e.g., a subject, with a stable composition of the present invention prior to an ischemic, hypoxic, or reperfusion injury or a disease insult. These methods can be used when an injury or disease with the potential to cause ischemia or hypoxia is scheduled or elected in advance, or is predicted in advance to likely occur. Examples include, but are not limited to, major surgery where blood loss may occur spontaneously or as a result of a procedure, cardiopulmonary bypass in which oxygenation of the blood may be compromised or in which vascular delivery of blood may be reduced (as in the setting of coronary artery bypass graft (CABG) surgery), or in the treatment of organ donors prior to removal of donor organs for transport and transplantation into a recipient in need of an organ transplant. Examples include, but are not limited to, medical conditions in which a risk of injury or disease progression is inherent (e.g., in the context of unstable angina, following angioplasty, bleeding aneurysms, hemorrhagic strokes, following major trauma or blood loss), or in which the risk can be diagnosed using a medical diagnostic test.

Relatedly, additional embodiments of the invention concern enhancing survivability and preventing irreversible tissue damage from blood loss or other lack of oxygenation to cells or tissue, such as from lack of an adequate blood supply. This may be the result of, for example, actual blood loss, or it may be from conditions or diseases that cause blockage of blood flow to cells or tissue, that reduce blood pressure locally or overall in an organism, that reduce the amount of oxygen that is carried in the blood, or that reduces the number of oxygen carrying cells in the blood. Conditions and diseases that may be involved include, but are not limited to, blood clots and embolisms, cysts, growths, tumors, anemia (including sickle cell anemia), hemophilia, other blood clotting diseases (e.g., von Willebrand, or ITP), and atherosclerosis. Such conditions and diseases also include those that create essentially hypoxic or anoxic conditions for cells or tissue in an organism because of an injury, disease, or condition.

In one embodiment, the present invention provides methods to enhance the survivability of or reduce or prevent injury or damage to biological material (e.g., a mammal or tissue or organ within a mammal) undergoing hemorrhagic shock or undergoing reperfusion, which include contacting the biological material at risk of or in a state of hemorrhagic shock with an effective amount of a stable composition as soon as practical, ideally within one hour of the injury. This method allows for the subject to be transported to a controlled environment (e.g., surgery), where the initial cause of the injury can be addressed, and then the patient can be brought back to normal function in a controlled manner. For this indication, the first hour after injury, referred to as the “golden hour,” is crucial to a successful outcome.

In various other embodiments, the methods of the present invention may be used in the treatment of neurodegenerative diseases associated with ischemia, hypoxia or reperfusion, in the treatment of hypothermia, in the treatment of hyperproliferative disorders, and in the treatment of immune disorders. In various other embodiments, the biological condition is any one or combination of the following: neurological disease, cardiovascular disease, metabolic disease, infectious disease, lung disease, genetic disease, autoimmune disease, and immune-related disease.

In certain embodiments, the methods of the present invention are used to enhance the survivability of ex vivo biological matter subjected to hypoxic or ischemic conditions, including, e.g., isolated cells, tissues and organs. Specific examples of such ex vivo biological material include platelets and other blood products, as well as tissues and organs to be transplanted.

In certain embodiments, the amount of or effective amount of a stable composition of the invention that is provided to biological material, e.g., a mammal or tissue therein, is about, at least, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mg, mg/kg, or mg/m², or any range derivable therein. Alternatively, the amount may be expressed as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mM or M, or any range derivable therein.

In particular embodiments of any of the methods of the present invention, a biological material, e.g., a subject, is treated with or contacted with an effective amount of a stable composition of the present invention, wherein said effective amount of about 0.01 mg/kg to about 20 mg/kg, about 0.05 mg/kg to about 10 mg/kg, about 0.1 mg/kg to about 5 mg/kg, about 0.5 mg/kg to about 2 mg/kg, about 0.5 mg/kg to about 1 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg or about 1.2 mg/kg. In particular embodiments, the stable composition comprises selenide, e.g., sodium selenide or another form of selenide in its −2 state.

In particular embodiments, an effective amount of a stable composition is administered to a biological material, e.g., a tissue or subject, prior to reperfusion, e.g., for at least one, two three, four, five or ten minutes of the ten minutes or thirty minutes immediately prior to reperfusion. In particular embodiments, this administration of the stable composition results in reduced reperfusion injury. In certain embodiments, the reperfusion injury is less than 80%, less than 70%, less than 60%, less than 50%, less than 40%, or less than 30% the reperfusion injury in the absence of treatment with the stable composition. In particular embodiments, the stable composition comprises a chalcogenide comprising a chalcogen e.g., sulfur or selenium, in a −2 valence state. In particular embodiments, the reduced form of a chalcogenide is H₂Se, Na₂Se, NaHSe, HSe—, H₂S, NaHS, Na₂S, or HS—. In related embodiments, the reduced form of a chalcogenide is produced by a method described herein.

In particular embodiments, treatment of a biological material, i.e., a subject, with a stable composition of the present invention following an injury, e.g., heart attack or stroke, results in a reduced injury, e.g., infarct size. In certain embodiments, the injury or infarct size is less than 80%, less than 70%, less than 60%, less than 50%, less than 40%, or less than 30% of the severity or infarct size that occurs in the absence of treatment with the stable composition. In particular embodiments, the stable composition comprises a chalcogenide comprising a chalcogen e.g., sulfur or selenium, in a −2 valence state. In particular embodiments, the reduced form of a chalcogenide is H₂Se, Na₂Se, NaHSe, HSe—, H₂S, NaHS, Na₂S, or HS—. In related embodiments, the reduced form of a chalcogenide is produced by a method described herein.

In particular embodiments, treatment of a biological matter, e.g., a subject within 10 minutes, within 30 minutes, within one hour, or within two hours or a heart attack, with a stable composition results in a decrease in heart damage, or an increase in fractional shortening or left ventricular function, as compared to in the absence of treatment with the stable composition. In particular embodiments, the decrease in heart damage is at least 10%, at least 20%, at least 30%, at least 40%, or at least 50%, as compared to in the absence of treatment with the stable composition. Decrease in heart damage may be detected by assessing levels of the heart specific protein, cardiac troponin I, in the blood of a subject after treatment. In particular embodiments, the increase in fractional shortening or left ventricular function is at least 20%, at least 30%, at least 40%, or at least 50%, as compared to in the absence of treatment with the stable composition. In particular embodiments, the stable composition comprises a chalcogenide comprising a chalcogen e.g., sulfur or selenium, in a −2 valence state. In particular embodiments, the reduced form of a chalcogenide is H₂Se, Na₂Se, NaHSe, HSe—, H₂S, NaHS, Na₂S, or HS—. In related embodiments, the reduced form of a chalcogenide is produced by a method described herein.

In certain embodiments, the present invention includes a method of reducing inflammation in the heart of a subject, e.g., a mammal, heart attack, by administering to the subject a stable composition of the present invention. In certain embodiments, the decrease in heart inflammation is at least 10%, at least 20%, at least 30%, at least 40%, or at least 50%, as compared to in the absence of treatment with the stable composition. Inflammation may be measured as described in the accompanying examples. In particular embodiments, the stable composition comprises a chalcogenide comprising a chalcogen e.g., sulfur or selenium, in a −2 valence state. In particular embodiments, the reduced form of a chalcogenide is H₂Se, Na₂Se, NaHSe, HSe—, H₂S, NaHS, Na₂S, or HS—. In related embodiments, the reduced form of a chalcogenide is produced by a method described herein.

In various embodiments of methods of the present invention, a biological material, e.g., an organ, a subject, or a tissue therein, is exposed to a stable composition of the current invention for about, at least, at least about, or at most about 30 seconds, 1, 2, 3, 4, 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days or more, and any range or combination therein.

Furthermore, when administration is intravenous, it is contemplated that the following parameters may be applied. In certain embodiments, the flow rate is about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 gtts/min or μgtts/min, or any range derivable therein. In some embodiments, the amount of the stable composition is specified by volume, depending on the concentration of the stable chalcogenide composition. In certain embodiments, an amount of time may be about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, and/or 1, 2, 3, 4, 5, 6, 7, 8, 9, 10, 11, 12 months, or any range derivable therein.

In certain embodiments, volumes of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mls or liters, or any range therein, may be administered overall or in a single session.

According to various embodiments of the methods of the present invention, a biological material is provided with a stable composition of the invention, e.g., intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, intraocularly, subcutaneously, subconjunctival, intravesicularly, mucosally, intrapericardially, intraumbilically, intraocularally, orally, topically, locally, by injection, by infusion, by continuous infusion, by absorption, by adsorption, by immersion, by localized perfusion, via a catheter, or via a lavage. In particular embodiments, it is provided parenterally, e.g., intravenously, or by inhalation. “Parenteral” refers to any route of administration of a substance other than via the digestive tract. A therapeutic agent is an active agent.

### D. Delivery Devices

In additional embodiments, the present invention includes a drug delivery device designed to limit, prevent or inhibit oxidation of a reduced form of an active substance, such as, e.g., a reduced form of a chalcogenide. In specific embodiments, the drug delivery device maintains a reduced form of an active agent or active substance, e.g., therapeutic agent, in its reduced form. In particular embodiments, the device comprises the reduced form of a chalcogenide, such as selenide or sulfide, for example. In specific embodiments, the drug device comprises a stable composition of the present invention.

Manufacturer-prepared, premixed ready-to-use products represent a useful approach to intravenous drug safety, since they remove error associated with measuring and diluting intravenous drugs. Accordingly, in certain embodiments, the present invention includes a drug delivery device for administration of a ready-to-use product comprising a reduced form of a therapeutic agent. In particular embodiments, the reduced form of therapeutic agent is a reduced form of a chalcogenide, e.g., selenide or sulfide.

In addition, it is understood that traditional methods for delivering therapeutic agents, which can involve injecting air into a vial comprising the therapeutic agent during the process of withdrawing the agent into a syringe or bag, may result in the undesired oxidation of a therapeutic agent that is in a reduced form. Accordingly, the present invention provides delivery devices that minimize or prevent contact of a therapeutic agent with oxygen during delivery to a subject.

In one embodiment, the present invention includes a drug delivery device, comprising:

a reservoir for containing a stable composition of the present invention; and

a fluid communicator, the fluid communicator configured to maintain at least 90% of the reduced form of active agent, e.g., chalcogenide, in the stable composition in reduced form during delivery to a subject. In particular embodiments, it is configured to maintain at least 95%, at least 96%, at least 97%, at least 98%, or at least 99% of the reduced form of active agent, e.g., chalcogenide, in reduced form during administration to a subject. In certain embodiments, the fluid communicator is in fluid communication with the reservoir. In other embodiments, it is can be placed into fluid communication with the reservoir.

In certain embodiments, the reservoir is oxygen impermeable, and/or it is formed of an oxygen impermeable polymer. In certain embodiments, the reservoir comprises an oxygen impermeable layer, e.g., at its inner surface, which may comprise: polyethylene (LDPE), polystyrene (PS), polyethylene (PE), polycarbonate (PC), polyvinyl acetate (PVAc), APET, polyvinly chloride (PVC), nylon 6 (Ny6), polyvinyl fluoride (PVF), polyvinylidene chloride (PVdC), polyacetonitrile (PAN), ethylene vinyl alcohol (EVOH), or Polyvinyl alcohol (PVA). In certain embodiments, the oxygen transmission coefficient of said polymer is less than 10⁻¹⁰ [cm³(STP)/cm/(cm²+s+Pa)]. In related embodiments, it is less than 10⁻⁹, less than 10⁻⁸, or less than 10⁻⁷ [cm³(STP)/cm/(cm²+s+Pa)]. In related embodiments, the reservoir includes multiple layers of oxygen impermeable polymers.

In particular embodiments, the reservoir includes a resealable port. A resealable port may be used for introducing a solution comprising a therapeutic agent, such as a stable composition of the present invention, into the reservoir. A resealable port may be used for coupling to the fluid communicator. In particular embodiments, the reservoir includes a plurality of resealable ports. In certain embodiments, the reservoir is a bottle, a bag, a tube, a vial, or a syringe. In particular embodiments, it is an intravenous bag (i.v. bag) or a syringe. In particular embodiments, it is a tubular member, the tubular member having a septum configured to hermetically seal the tubular member. In one embodiment, the tubular member is a Hungate tube.

In particular embodiments, the fluid communicator is configured to be fluidically coupled to the reservoir through a resealable port. In related embodiments, the fluid communicator is configured to intravenously deliver a therapeutic agent, e.g., a stable composition of the present invention, from the reservoir to a subject in need thereof. In certain embodiment, the fluid communicator includes at least one of a needle and a cannula.

In certain embodiments, the device, including the fluid communicator, is disposed in a reduced oxygen or oxygen-free environment. In particular embodiments, the reduced oxygen or oxygen-free environment is within a container, optionally wherein said container is a bag or a malleable container, e.g., that allows manipulation of components of the device through the bag, such as coupling of the fluid communicator to the reservoir in embodiments where they are not initially coupled. In particular embodiment, the container comprises one or more oxygen impermeable polymer, including any of those described above. In particular embodiments, the walls of the container comprise multiple layers of one or more oxygen impermeable polymers.

In particular embodiments, the device comprises in the reservoir a stable composition comprising a reduced form of an active agent, e.g., a reduced form of a chalcogenide. In certain embodiments, the reduced form of a chalcogenide is H₂Se, Na₂Se or NaHSe or a sulfide compound.

In particular embodiments, the device allows for delivery of the reduced form of an active agent, e.g., a reduced form of a chalcogenide, such as selenide or sulfide, wherein at least 90%, at least 95%, at least 96%, at least 97%, at least 98%, or at least 99% of the therapeutic agent is delivered to the subject in reduced form.

The present invention further includes a method for reducing injury or treating a disease in a subject by providing to said subject a stable composition of the invention, wherein said stable composition is provided to said mammal using the device of the present invention. In particular embodiments, the injury or disease is any of those described herein, including but not limited to an injury resulting from ischemia or reperfusion. In particular embodiments, the injury is an infarct caused by a heart attack or stroke. In other embodiments, the injury is caused by inflammation.

Relatedly, the present invention also includes a method of delivering a stable therapeutic composition in a reduced form to a patient in need thereof, the method comprising:

containing the therapeutic composition in a reservoir, the reservoir configured to maintain the therapeutic composition in a reduced form;

establishing fluid communication between the reservoir and the patient;

delivering a predetermined volume of the therapeutic composition from the reservoir to the patient in an environment substantially free of oxygen.

In particular embodiments, a device of the present invention comprises: (1) an i.v. bag comprising a stable composition of the present invention, wherein said i.v. bag is oxygen impermeable; and (2) a tubing, wherein said tubing is coupled at a first end of the tubing to the i.v. bag through a port in the i.v. bag, or wherein said tubing is capable of being coupled at a first end of the tubing to the i.v. bag through a port in the i.v. bag, wherein said tubing is coupled at a second end of the tubing to a needle or cannula, or wherein said tubing is capable of being coupled at a second end of the tubing to a needle or cannula, wherein said device is contained within a bag comprising a reduced oxygen or oxygen-free environment. In particular embodiments, the i.v. bag and/or the bag comprise one or more oxygen impermeable polymers described herein. In particular embodiments, the bag is flexible, such as to allow a user to couple the tubing to the i.v. bag and/or needle while the device remains sealed in the bag.

In particular embodiments, a device of the present invention comprises: (1) an i.v. bag comprising a stable composition of the present invention, wherein said i.v. bag is oxygen impermeable; and (2) a tubing, wherein said tubing is coupled at a first end of the tubing to the i.v. bag through a port in the i.v. bag, or wherein said tubing is capable of being coupled at a first end of the tubing to the i.v. bag through a port in the i.v. bag, wherein said tubing is coupled at a second end of the tubing to a needle or cannula, or wherein said tubing is capable of being coupled at a second end of the tubing to a needle or cannula, wherein said device is contained within a bag comprising a reduced oxygen or oxygen-free environment. In particular embodiments, the i.v. bag and/or the bag comprise one or more oxygen impermeable polymers described herein. In particular embodiments, the bag is flexible, such as to allow a user to couple the tubing to the i.v. bag and/or needle while the device remains sealed in the bag, and/to apply pressure to the i.v. bag to begin flow of the stable composition through the tubing towards or into the needle or cannula.

In particular embodiments, a device of the present invention comprises: (1) an syringe comprising a stable composition of the present invention, wherein said syringe is oxygen impermeable; and (2) a needle or cannula, wherein said needle or cannula is coupled to the syringe through a port in the syringe, or wherein said needle or cannula is capable of being coupled to the syringe through a port in the syringe, wherein said device is contained within a bag comprising a reduced oxygen or oxygen-free environment. In particular embodiments, the syringe and/or the bag comprise one or more oxygen impermeable polymers described herein. In particular embodiments, the bag is flexible, such as to allow a user to couple the needle or cannula to the syringe while the device remains sealed in the bag, and/to apply pressure on the syringe to begin flow of the stable composition into the needle or cannula.

In particular embodiments, the device comprises a therapeutically effective amount of the therapeutic agent or stable composition.

### EXAMPLES

### Example 1

### Sodium Selenide Improves Myocardial Infarction Outcome

This example demonstrates that sodium selenide (NaHSe) improves outcome in a mouse model of heart attack to a degree that exceeds that seen by any other method. The basis for this conclusion comes from several independent lines of evidence including reduction in infarct size, reduction in a blood marker of lysed cardiomyocytes, histological measures, and improved heart function (e.g., ejection fraction and fractional shortening) as judged by echocardiography. The results are summarized below.

### Heart Attack Model

For the myocardial infarction model, open chest ligation of the left descending coronary artery (LAD ligation) was performed in mice. At 55 minutes post initiation of ischemia, either vehicle or test agent was injected into the femoral vein of each mouse. The heart muscle was reperfused by releasing the ligature at 60 minutes post initiation of ischemia. Mice were then sacrificed at either 2 hours or 2 days post reperfusion for infarct size measurement and blood analysis or heart function assays, respectively, as described below.

### Infarct Size

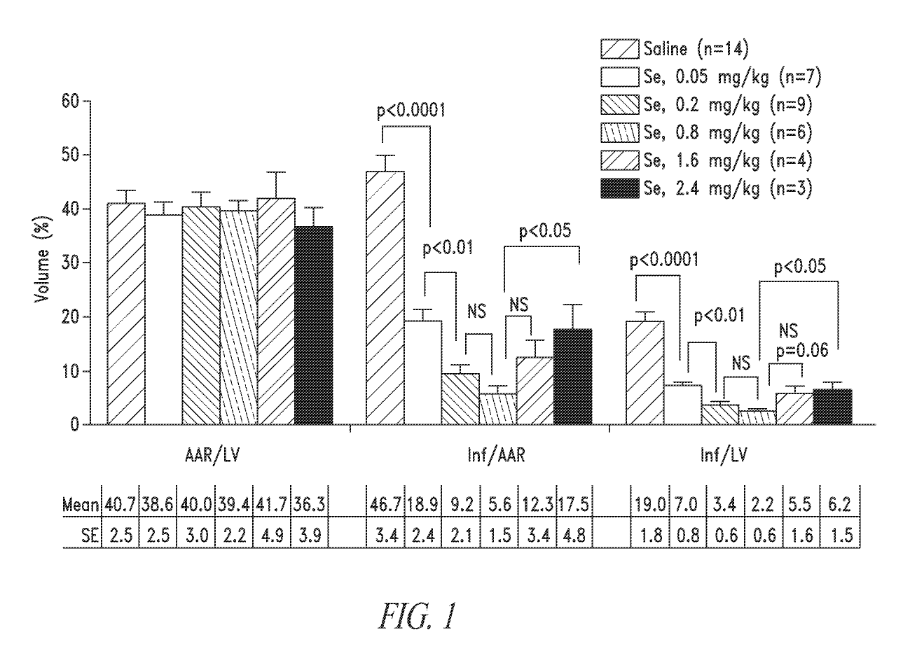

In order to examine the effect of NaHSe on myocardial infarct size, mice were subjected to LAD ligation, and after 2 hours of reperfusion, infarct size was measured by morphometry (FIG. 1). Six groups of mice were utilized in this study, including a saline vehicle group and five test groups that received 50, 200, 800, 1600 and 2400 μg/kg of NaHSe. To be certain that all animals in the study were treated in a similar way, the area at risk (AAR) as a proportion of the left ventricle was determined (FIG. 1, left panel).

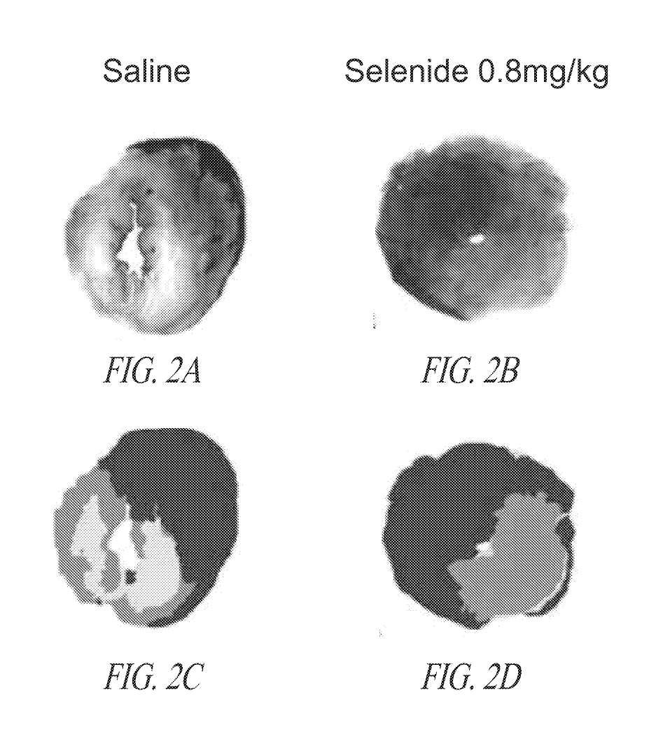

Area at risk and infarct size (Inf) were measured using the specific dyes TTC and Evans blue. An example of the result of this staining is shown in FIG. 2. Processed images were generated in an unbiased way by setting constant thresholds and allowing Photoshop® to generate the images that were quantitated to generate FIG. 1.

These results show that there were statistically significant dose dependent decreases in infarct size when comparing the saline group to all five test groups that received increasing concentrations of NaHSe between 50 and 2,400 μg/kg (FIG. 1, middle panel). In this study, the optimal dose appeared to be 800 μg/kg, which resulted in an 88% reduction in infarct size in comparison to vehicle alone.

### Blood Analysis

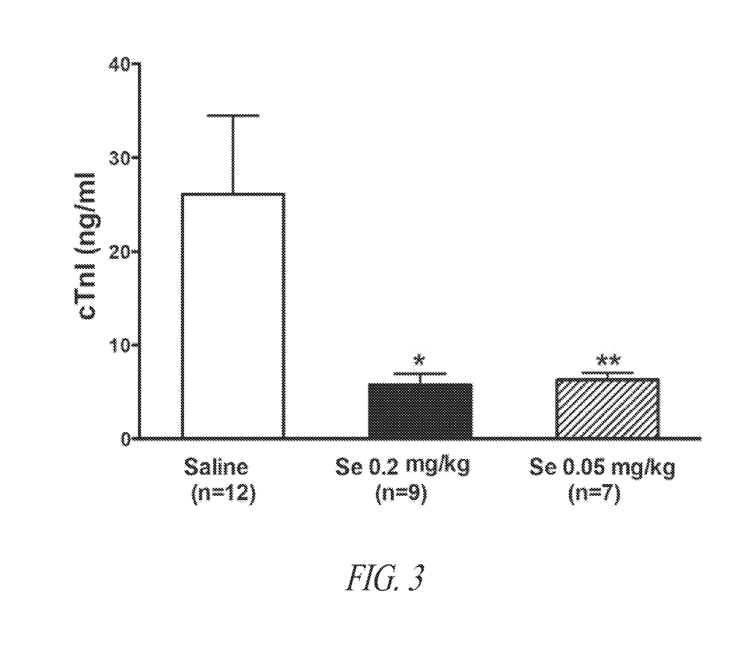

To provide an independent means of testing for improvement, the level of a heart specific protein, cardiac troponin I, was assessed in the blood of the animals after the 2 hour reperfusion period. Five minutes prior to reperfusion, three groups of mice were given 200 μg/kg NaHSe, 50 μg/kg NaHSe, or saline by I.V. injection through the femoral vein. At the end of the 2 hour reperfusion, blood was collected and plasma cardiac troponin I was measured by ELISA. The 50 μg/kg and 200 μg/kg test groups showed a statistically significant decrease in cardiac troponin I compared to the saline control indicating that NaHSe administration decreases heart damage (FIG. 3). Selenide treatment significantly reduced the level of plasma cardiac troponin I in myocardial infarct hearts.

### Heart Function

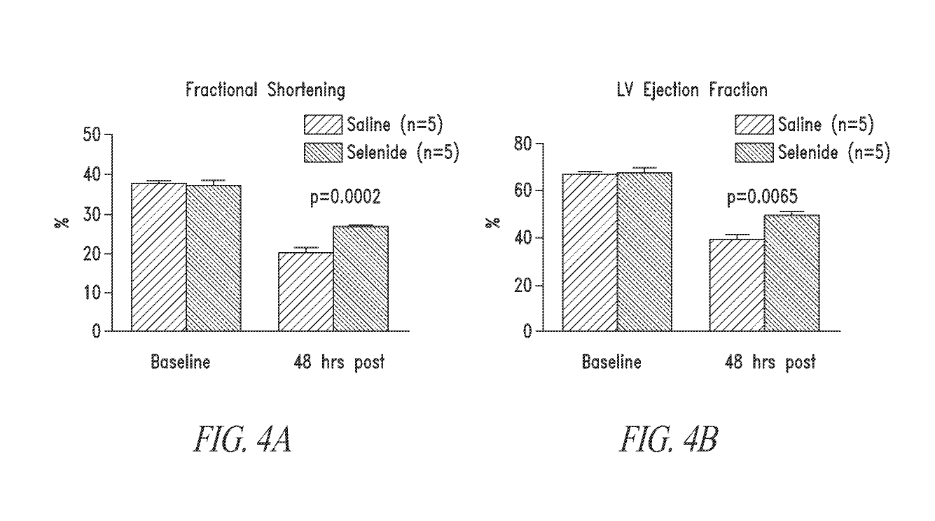

To further test the hypothesis that NaHSe improves outcome following myocardial infarction, mice were survived for 2 days post the heart attack procedure and then subjected to echocardiography. Using this technique, it was shown that administration of 800 μg/kg of NaHSe resulted in a statistically significant improvement in both fractional shortening and left ventricular ejection fraction (FIG. 4).

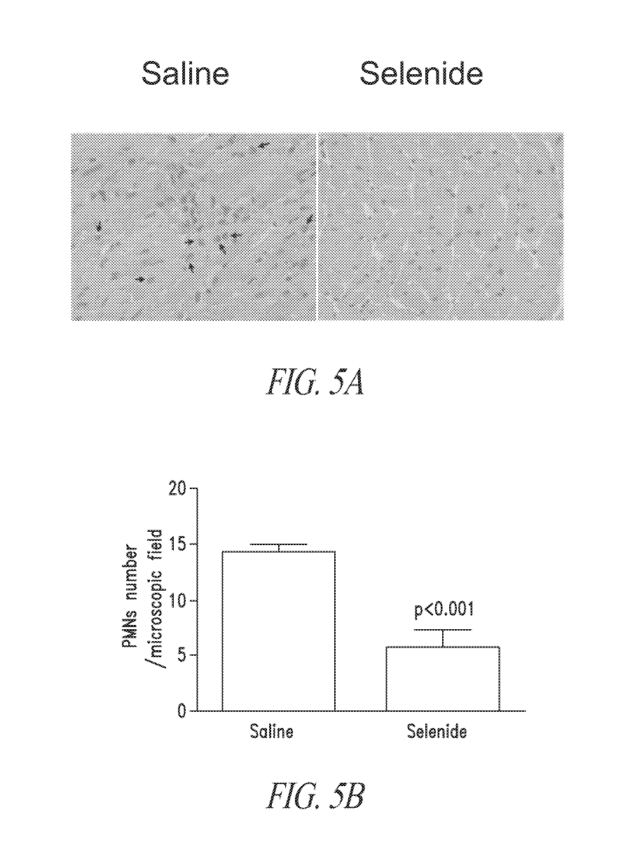

In order to determine whether NaHSe decreased inflammation in the hearts of animals following the heart attack procedure, neutrophils were counted in sections of hearts of saline and NaHSe treated animals. These results showed that NaHSe administration significantly reduced the accumulation of neutrophils in the myocardium following heart attack (FIG. 5).

### Sodium Selenite in Comparison to Sodium Selenide

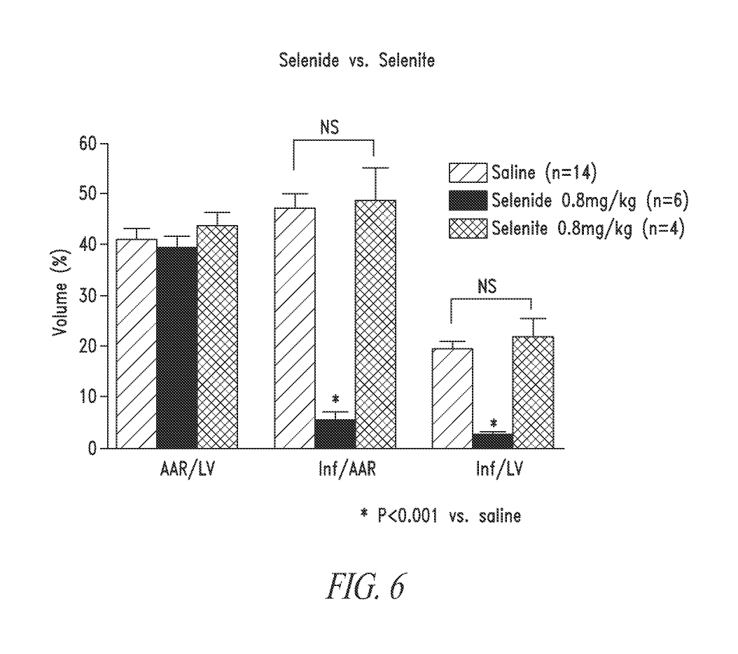

Selenium is required for the synthesis of specific proteins critical for the degradation of hydrogen peroxide. Given that hydrogen peroxide is widely thought to cause damage in the cells of critical care patients, physicians have been interested in administrating selenium to patients to improve clinical outcome. Before the work by the inventors described herein, it was not possible to make the substrate for selenoprotein synthesis, selenide, and selenite has been used as a result. There have been 21 critical care disease trials using selenite, all of which failed (trials.gov). Therefore it was desirable to compare sodium selenite with sodium selenide in the present model. Three groups of mice were given saline vehicle, 800 μg/kg NaHSe, or 800 μg/kg sodium selenite at reperfusion in the heart attack model. The results show that sodium selenite provided no benefit (FIG. 6).

### Selenium Levels Following Heart Attack

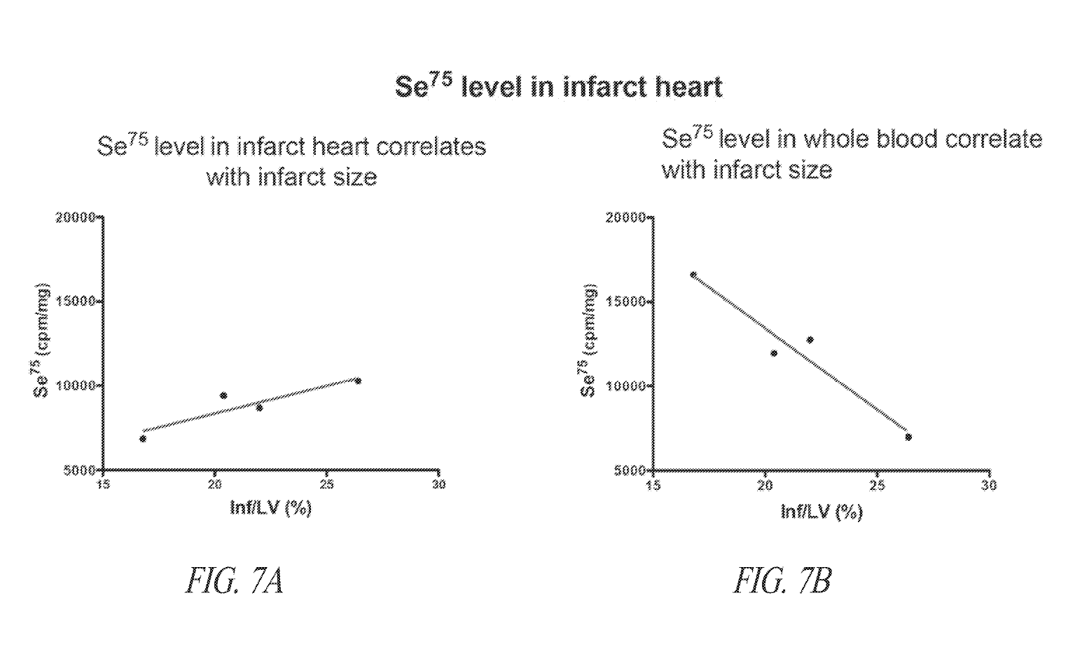

Decades of work by others shows that selenium levels decrease in the blood of patients that experience many different diseases including sepsis, burns, liver failure, cardiac arrest, cardiac bypass surgery, and chronic heart failure as well as patients admitted to intensive care units for all causes. For this reason, the hypothesis that selenium levels in the heart and the blood may change according to the size of the infarct was tested. The heat attack model was conducted using animals that had been given a tracer dose of radioactive selenium intravenously 5 days prior to the heart attack. It was found that selenium levels increased in the heart with increasing infarct size and that selenium levels in the blood decreased with increasing infarct size (FIG. 7). These results suggested that selenium is moving from the blood to the heart during a heart attack in the mouse model.

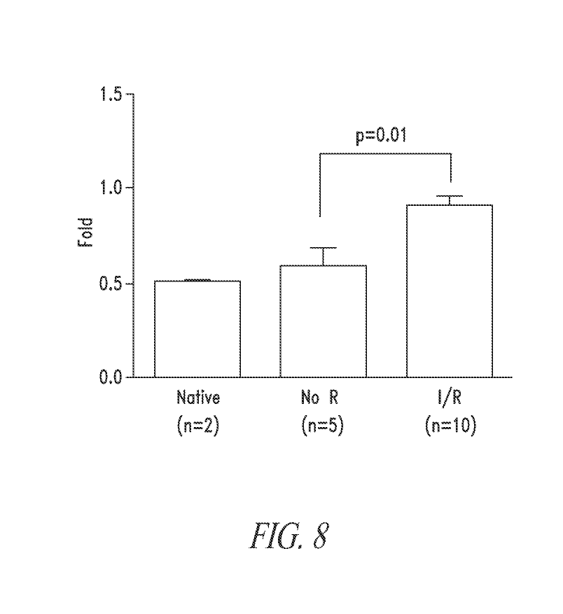

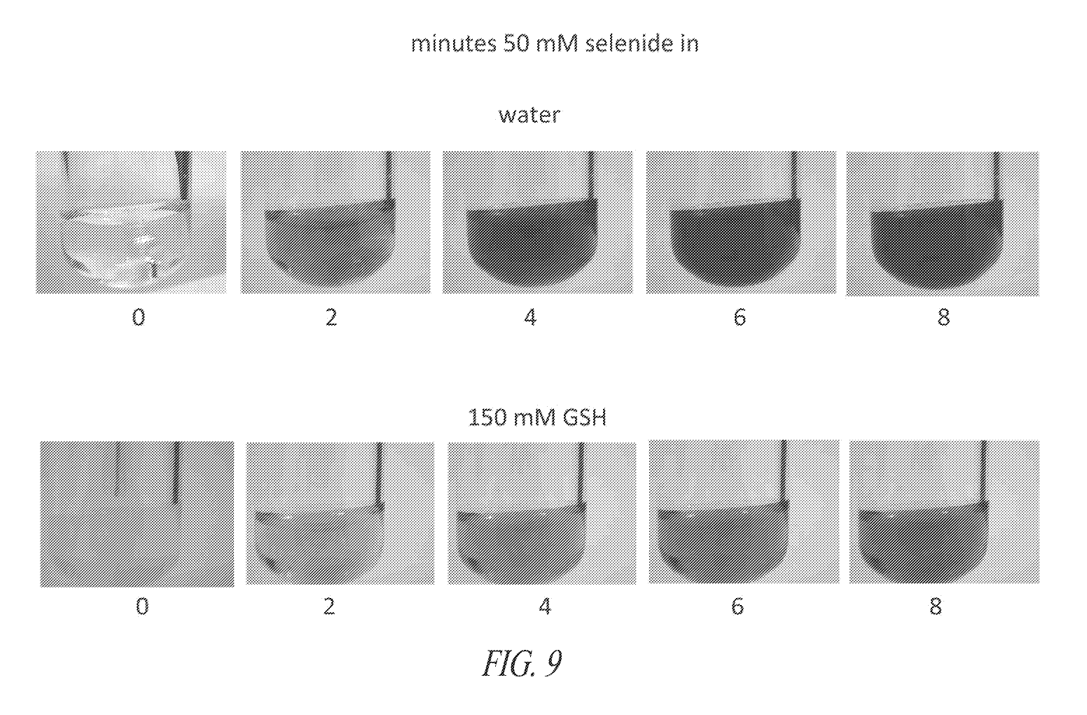

In order to understand when the selenium was accumulating in the heart during the procedure, hearts were collected either immediately after the ischemic period or after the reperfusion period from animals labeled with radioactive selenium as described above. The relative measure of radioactive selenium in heart tissue from three groups of mice (native, 60 minutes of ischemia with no reperfusion, and 60 min of ischemia with 2 hours of reperfusion) was determined. It was found that there was a reperfusion-dependent accumulation of radioactive selenium in the heart in animals that went through the heart attack procedure (FIG. 8). Upon further analysis it was found that selenium was diminished in infarct tissue compared to undamaged tissue (FIG. 9).

### Selenoprotein Synthesis

The sole function of selenium is to be a substrate for the synthesis of selenoproteins. The chemical form of selenium required for the synthesis of selenoproteins is selenide. Some selenoproteins, such as glutathione peroxidase, degrade hydrogen peroxide, and hydrogen peroxide is believed to be responsible for tissue damage in heart attacks.

Therefore, the hypothesis that the therapeutic value of sodium selenide is its accumulation in cardiomycytes during reperfusion for the synthesis of selenoproteins, such as glutathione peroxidase, was tested. Heart sections from animals that were saline treated or NaHSe (800 μg/kg) treated were stained with an antibody specific for the selenoprotein glutathione peroxidase 2 (GPX-2). There was a striking increase in the staining with this antibody in sections taken from animals that were administered NaHSe when compared to sections from saline and sham (no heart attack) animals (FIG. 10). These results suggest the benefit from sodium selenide was conferred through enhancing the expression of selenoproteins.

### Example 2

### Glutathione Stabilizes Selenide

To demonstrate that glutathione prevents selenide from oxidation, solutions of 50 mM selenide were prepared in either water or 150 mM GSH and observed for the 8 minutes immediately following preparation. In FIG. 9, the oxidized forms of selenide appear dark in the solutions, with the samples in glutathione clearly showing reduced levels of oxidation at each time point.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention.

We claim:

1. A method of producing a stable composition comprising a reduced form of a chalcogenide, said method comprising:
admixing a chalcogen, or an acid of a chalcogen, and a reducing agent in a reduced oxygen environment under conditions and for a time period sufficient to allow oxidation of a majority of the reducing agent and reduction of a majority of the chalcogen,
thereby producing a stable composition comprising a reduced form of a chalcogenide.

2. The method of claim 1, wherein said chalcogen is sulfur or selenium.

3. The method of claim 1, wherein said acid of a chalcogen is selenious acid or sodium selenite or elemental selenium.

4. The method of any one of claims 1-3, wherein said reducing agent has a reduction potential (E°) less than or equal to about 0.4 V.

5. The method of any one of claims 1-4, wherein said reducing agent is sodium borohydride (NaBH₄).

6. The method of any one of claims 1-5, wherein the molar ratio of the reducing agent to the chalcogen, or the acid of a chalcogen, is about 5:1 to about 0.5:1.

7. The method of claim 6, wherein the molar ratio of the reducing agent to the chalcogen, or the acid of a chalcogen, is about 2:1.

8. The method of any one of claim 6 or 7, wherein said reducing agent is sodium borohydride, said chalcogen is sulfur or selenium, and said acid of a chalcogen is selenious acid.

9. The method of claim 8, wherein said reducing agent is sodium borohydride, said chalcogen is selenium, and said molar ratio of sodium borohydride to selenium is about 2:1.

10. The method of claim 9, wherein said selenium is present in an amount of about 1 mM to 1M (79 mg/L to 79 g/L).

11. The method of claim 9, wherein said sodium borohydride is present in a 1M solution in water.

12. The method of any one of claims 1-11, wherein said method is performed in an oxygen-free environment.

13. The method of any one of claims 1-12, wherein said method is performed under nitrogen.

14. The method of claim 13, wherein said nitrogen is perfused into said reduced oxygen environment.

15. The method of claim 14, wherein said nitrogen is perfused at a rate of about 100 cc/min.

16. The method of any one of claims 1-15, wherein said reduced oxygen environment is a container having an oxygen-free environment.

17. The method of claim 16, wherein said container is a tube or a vial.

18. The method of claim 16 or claim 17, wherein said container comprises a closable port of entry.

19. The method of claim 18, wherein said container is a sealable tube comprising a rubber septum, e.g., a Hungate tube.

20. The method of claim 19, wherein the perfusion of nitrogen into and out of the tube occurs via two needles that pass through the septum, wherein one of the two needles is used as the port to bring nitrogen into the tube, and wherein the second of the two needles is used to take nitrogen out of the tube.

21. The method of any one of claims 1-20, wherein said conditions comprise a temperature of about room temperature.

22. The method of any one of claims 1-21, further comprising heating the admixture after said time period.

23. The method of any one of claims 1-22, wherein said time period ends when the admixture solution appears visibly clear.

24. The method of claim 22, wherein said heating is continued until any observed bubbling ceases.

25. The method of claim 22-24, further comprising cooling the admixture after said heating.

26. The method of claim 25, wherein said cooling is performed by placing the admixture on ice.

27. The method of claim 25 or claim 26, wherein said cooling is continued until sodium borate precipitates from the admixture solution.

28. The method of claim 27, further comprising centrifuging said admixture solution to separate a supernatant from the precipitated sodium borate, and removing said supernatant.

29. The method of claim 28, wherein said supernatant comprises said stable composition comprising said reduced chalcogenide.

30. The method of any one of claims 1-29, wherein at least 90% of said chalcogenide is present in a reduced form for at least 1 hour.

31. The method of claim 8, further comprising acidifying the admixture with an acid, wherein said acid is reducing and not volatile, and bubbling hydrogen selenide gas through a solution, wherein said solution has a pH greater than 3.9.

32. The method of claim 31, wherein said acid is phosphorous acid and said solution is phosphate buffered saline (PBS).

33. The method of any one of claims claim 1-32, wherein said reduced form of the chalcogenide is in the minus 2 oxidation state.

34. The method of any one of claims 1-32, wherein the reduced form of the chalcogenide is H₂Se, Na₂Se, NaHSe, or HSe-anion.

35. A method of producing a stable composition comprising a reduced chalcogenide, said method comprising:
admixing elemental selenium (Se) or sulfur (S) and sodium hydride in a solution comprising mineral oil or tetrahydrofuran (THF),
thereby producing a stable composition comprising sodium hydroselenide or sodium sulfide.

36. The method of claim 35, further comprising adding water to the admixture solution, thereby removing sodium hydride.

37. The method of claim 35or claim 36, wherein said solution comprises mineral oil, wherein said method further comprises removing the aqueous phase of the admixture solution, wherein said reduced chalcogenide is present in said aqueous phase.

38. The method of claim 35 or claim 36, wherein said solution comprises THF, wherein said method further comprises removing said THF by boiling said admixture solution at about 70° C.

39. A stable composition comprising a reduced form of a chalcogenide, wherein at least 90% of said chalcogenide in said composition is present in said reduced form for at least one hour when stored at room temperature.

40. The stable composition of claim 39, further comprising a pharmaceutically acceptable carrier, diluent or excipient.

41. The stable composition of claim 39 or claim 40, wherein said reduced form of a chalcogenide comprises a chalcogen in its −2 oxidation state, wherein said reduced form of chalcogenide is optionally H₂Se, Na₂Se, NaHSe, or HSe-anion.

42. A stable composition comprising a reduced form of a chalcogenide produced by a method of any one of claims 1-38.

43. The stable composition of any one of claims 39-42, wherein said stable composition further comprises one or more of a reducing agent, a tonicity agent, a stabilizer, a surfactant, a lycoprotectant, a polyol, an antioxidant, or a preservative.

44. The stable composition of claim 43, wherein said elemental chalcogen is selenium or sulfur.

45. The stable composition of any one of claims 39-44, wherein said stable composition further comprises a solvent.

46. The stable composition of claim 45, where said solvent is water.

47. A device for maintaining a compound in a reduced form, said device comprising an oxygen impermeable container.

48. The device of claim 47, wherein said container is glass.

49. The device of claim 47, wherein the walls of said container comprise an oxygen impermeable polymer.

50. The device of claim 49, wherein said polymer is selected from the group consisting of: silicon rubber, natural rubber, low density poly ethylene (LDPE), polystyrene (PS), polyethylene (PE), polycarbonate (PC), polyvinyl acetate (PVAc), amorphous polyethylene terephthalate (APET), polyvinly chloride (PVC), nylon 6 (Ny6), polyvinyl fluoride (PVF), polyvinylidene chloride (PVdC), polyacetonitrile (PAN), ethylene vinyl alcohol (EVOH), and polyvinyl alcohol (PVA).

51. The device of 49 or claim 50, wherein the oxygen transmission coefficient of said polymer is less than 10⁻¹⁰ [cm³(STP)/cm/(cm²+s+Pa)].

52. The device of any one of claims 47-51, wherein the walls of said container comprise multiple layers of one or more oxygen impermeable polymers.

53. The device of any one of claims 47-52, wherein said container comprises a closable port of entry.

54. The device of any one of claims 47-53, wherein said container comprises two or more closable ports of entry.

55. The device of claim any one of claims 47-54, wherein said container is a bottle, a bag, a tube, a vial, or a syringe.

56. The device of claim 55, wherein said device is an intravenous bag or a syringe.

57. The device of any one of claims 47-55, wherein said container is a sealable tube comprising a rubber septum, e.g., a Hungate tube.

58. The device of any one of claims 47-57, wherein said device further comprises a delivery means coupled to the container through a closable port of entry.

59. The device of claim 58, wherein said delivery means is configured to intravenously deliver a solution from the container to a subject in need thereof.

60. The device of claim 58 or claim 59, wherein said delivery means comprises a needle or cannula.

61. The device of any one of claims 58-60, wherein said delivery means comprises a reduced oxygen environment.

62. The device of any one of claims 47-61, wherein said compound is a chalcogenide.

63. The device of claim 62, wherein said reduced form of a chalcogenide is H₂Se, Na₂Se, NaHSe, or HSe-anion.

64. The device of any one of claims 47-63, wherein said device comprises the stable composition of any one of claims 39-46.

65. A method for reducing injury or treating a disease in a subject comprising providing to said subject the stable composition of any one of claims 39-46.

66. The method of claim 65, wherein said stable composition is provided to said subject using the device of any one of claims 47-64.

67. The method of claim 65 or claim 66, wherein said injury is the result of ischemia or reperfusion.

68. The method of any one of claims 65-67, wherein said injury is an infarct caused by a heart attack or stroke.

69. The method of claim 65 or claim 66, wherein said injury is caused by inflammation, heart attack, coronary bypass surgery, ischemia, gut ischemia, liver ischemia, kidney ischemia, stroke, traumatic brain injury, limb ischemia, eye ischemia, sepsis, smoke, burn, or acute lung injury.

70. A drug delivery device, comprising:
a reservoir for containing a stable composition of any one of claims 39-46 in a reduced form; and
a fluid communicator in fluid communication with the reservoir, the fluid communicator configured to maintain at least 90% of the composition in reduced form during delivery to a patient.

71. The device of claim 70, wherein the reservoir is formed of an oxygen impermeable polymer.

72. The device of claim 71, wherein said polymer is selected from the group consisting of: silicon rubber, natural rubber, low density polyethylene (LDPE), polystyrene (PS), polyethylene (PE), polycarbonate (PC), polyvinyl acetate (PVAc), APET, polyvinly chloride (PVC), nylon 6 (Ny6), polyvinyl fluoride (PVF), polyvinylidene chloride (PVdC), polyacetonitrile (PAN), ethylene vinyl alcohol (EVOH), and Polyvinyl alcohol (PVA).

73. The device of claim 71 or claim 72, wherein the oxygen transmission coefficient of said polymer is less than 10⁻¹⁰ [cm³(STP)/cm/(cm²+s+Pa)], wherein Pa=pascals; STP=standard temperature and pressure (25 degrees centrigrade and pressure 1 atmosphere); and s=second.

74. The device of any one of claims 70-73, wherein the reservoir includes multiple layers of oxygen impermeable polymers.

75. The device of any one of claims 70-74, wherein said reservoir includes a resealable port.

76. The device of any one of claims 70-75, wherein said reservoir includes a plurality of resealable ports.

77. The device of claim any one of claims 70-76, wherein said reservoir is a bottle, a bag, a tube, a vial, or a syringe.

78. The device of claim 77, wherein said device is an intravenous bag or a syringe.

79. The device of any one of claims 70-77, wherein said reservoir is a tubular member, the tubular member having a septum configured to hermetically seal the tubular member.

80. The device of claim 79, wherein the tubular member is a Hungate tube.

81. The device of any one of claims 70-80, wherein said fluid communicator is configured to be fluidically coupled to the reservoir through a resealable port.

82. The device of claim 81, wherein said fluid communicator is configured to intravenously deliver the composition from the reservoir to a subject in need thereof.

83. The device of claim claim 81 or claim 82, wherein said fluid communicator includes at least one of a needle and a cannula.

84. The device of any one of claims 81-83, wherein said fluid communicator is disposed in a reduced oxygen or oxygen-free environment.

85. The device of any one of claims 70-84, wherein said composition comprises a reduced form of a chalcogenide.

86. The device of claim 85, wherein said reduced form of a chalcogenide is H₂Se, Na₂Se or NaHSe.

87. The device of any one of claims 70-86, wherein said device is disposed in a reduced oxygen or oxygen-free environment.

88. The device of claim 87, wherein said reduced oxygen or oxygen-free environment is within a container, optionally wherein said container is a bag.

89. The device of claim 88, wherein said container comprises an oxygen impermeable polymer.

90. The device of claim 89, wherein said polymer is selected from the group consisting of: silicon rubber, natural rubber, low density poly ethylene (LDPE), polystyrene (PS), polyethylene (PE), polycarbonate (PC), polyvinyl acetate (PVAc), amorphous polyethylene terephthalate (APET), polyvinly chloride (PVC), nylon 6 (Ny6), polyvinyl fluoride (PVF), polyvinylidene chloride (PVdC), polyacetonitrile (PAN), ethylene vinyl alcohol (EVOH), and polyvinyl alcohol (PVA).

91. The device of 89 or claim 90, wherein the oxygen transmission coefficient of said polymer is less than 10⁻¹⁰ [cm³(STP)/cm/(cm²+s+Pa)].

92. The device of any one of claims 88-91, wherein the walls of said container comprise multiple layers of one or more oxygen impermeable polymers.

93. A method for reducing injury in a mammal by providing to said mammal the stable composition of any one of claims 39-46.

94. The method of claim 93, wherein said stable composition is provided to said mammal using the device of any one of claims 70-92.

95. The method of claim 93 or claim 94, wherein said injury is the result of ischemia or reperfusion.

96. The method of any one of claims 93-95, wherein said injury is an infarct caused by a heart attack or stroke.

97. The method of claim 93 or claim 94, wherein said injury is caused by inflammation.

98. A method of delivering a stable therapeutic composition in a reduced form to a patient in need thereof, the method comprising:
containing the therapeutic composition in a reservoir, the reservoir configured to maintain the therapeutic composition in a reduced form;
establishing fluid communication between the reservoir in the patient;
delivering a predetermined volume of the therapeutic composition from the reservoir to the patient in an environment substantially free of oxygen.

99. The method of any one of claims 39-46, wherein said method comprises admixing glutathione with the chalcogen and reducing agent.

100. The method of any one of claims 39-46, wherein said method comprises adding glutathione to the stable composition.

101. The composition, method, or device of any of the preceding claims, wherein the stable composition comprises glutathione at a concentration of about 1.5 μM to about 10 M, about 15 μM to about 1 M, about 150 μM to about 1 M, about 1.5 mM to about 1 M, about 10 mM to about 500 mM, about 10 mM to about 250 mM, or about 100 mM, about 120 mM, about 150 mM, about 170 mM, or about 200 mM.
